SEQUENCE LISTING (90 sequences)

SEQ ID NO: 1 - protein, 6 aa, Artificial Sequence
  protease recognition site
AKRRSQ

SEQ ID NO: 2 - protein, 13 aa, Artificial Sequence
  protease recognition site
SPLAQAVKSSSRK

SEQ ID NO: 3 - protein, 32 aa, Artificial Sequence
  protease recognition site
GSDMELPLPRNITEGEARGSVILTVKPIFEEF

SEQ ID NO: 4 - protein, 20 aa, Artificial Sequence
  protease recognition site
GSKTEEISEVNLDAEFRHDS

SEQ ID NO: 5 - protein, 7 aa, Artificial Sequence
  serine protease splicing target
EGRIVEG

SEQ ID NO: 6 - protein, 7 aa, Artificial Sequence
  serine protease splicing target
RFKIIGG

SEQ ID NO: 7 - protein, 7 aa, Artificial Sequence
  serine protease splicing target
DDKIVGG

SEQ ID NO: 8 - protein, 7 aa, Artificial Sequence
  serine protease splicing target
LSRIVNG

SEQ ID NO: 9 - protein, 7 aa, Artificial Sequence
  serine protease splicing target
VYRVVGE

SEQ ID NO: 10 - protein, 7 aa, Artificial Sequence
  serine protease splicing target
AGKSNGE

SEQ ID NO: 11 - protein, 7 aa, Artificial Sequence
  serine protease splicing target
GIRSFRF

SEQ ID NO: 12 - protein, 7 aa, Artificial Sequence
  serine protease splicing target
PQRIVGG

SEQ ID NO: 13 - protein, 8 aa, Artificial Sequence
  serine protease splicing target
DFTRVVGG

SEQ ID NO: 14 - protein, 8 aa, Artificial Sequence
  serine protease splicing target
NLTRIVGG

SEQ ID NO: 15 - protein, 8 aa, Artificial Sequence
  serine protease splicing target
SMTRVVGG

SEQ ID NO: 16 - protein, 8 aa, Artificial Sequence
  serine protease splicing target
IKPRIVGG

SEQ ID NO: 17 - protein, 8 aa, Artificial Sequence
  serine protease splicing target
TSTRIVGG

SEQ ID NO: 18 - protein, 7 aa, Artificial Sequence
  serine protease splicing target
PGRVVGG

SEQ ID NO: 19 - protein, 7 aa, Artificial Sequence
  serine protease splicing target
AGEIIGG

SEQ ID NO: 20 - protein, 4 aa, Artificial Sequence
  caspase target splicing site
YEVD

SEQ ID NO: 21 - protein, 4 aa, Artificial Sequence
  caspase target splicing site
WEHD

SEQ ID NO: 22 - protein, 4 aa, Artificial Sequence
  caspase target splicing site
LEVD

SEQ ID NO: 23 - protein, 4 aa, Artificial Sequence
  caspase target splicing site
WVAD

SEQ ID NO: 24 - protein, 5 aa, Artificial Sequence
  caspase target splicing site
VDVAD

SEQ ID NO: 25 - protein, 4 aa, Artificial Sequence
  caspase target splicing site
DEHD

SEQ ID NO: 26 - protein, 5 aa, Artificial Sequence
  caspase target splicing site
LDESD

SEQ ID NO: 27 - protein, 4 aa, Artificial Sequence
  caspase target splicing site
IETD

SEQ ID NO: 28 - protein, 4 aa, Artificial Sequence
  caspase target splicing site
DMQC

SEQ ID NO: 29 - protein, 4 aa, Artificial Sequence
  caspase target splicing site
LEHD

SEQ ID NO: 30 - protein, 4 aa, Artificial Sequence
  caspase target splicing site
LEAD

SEQ ID NO: 31 - protein, 4 aa, Artificial Sequence
  caspase target splicing site
VEID

SEQ ID NO: 32 - protein, 4 aa, Artificial Sequence
  caspase target splicing site
VEHD

SEQ ID NO: 33 - protein, 4 aa, Artificial Sequence
  caspase target splicing site
VKMD

SEQ ID NO: 34 - protein, 4 aa, Artificial Sequence
  caspase target splicing site
VNLD

SEQ ID NO: 35 - protein, 4 aa, Artificial Sequence
  caspase target splicing site
DEVD

SEQ ID NO: 36 - protein, 4 aa, Artificial Sequence
  caspase target splicing site
LETD

SEQ ID NO: 37 - protein, 4 aa, Artificial Sequence
  caspase target splicing site
IEAD

SEQ ID NO: 38 - protein, 4 aa, Artificial Sequence
  caspase target splicing site
AEVD

SEQ ID NO: 39 - protein, 8 aa, Artificial Sequence
  SYNTHESIZED
GANPNAAG

SEQ ID NO: 40 - protein, 5 aa, Artificial Sequence
  SYNTHESIZED
KGGGK

SEQ ID NO: 41 - protein, 4 aa, Artificial Sequence
  SYNTHESIZED
KGGK

SEQ ID NO: 42 - protein, 4 aa, Artificial Sequence
  SYNTHESIZED
KAAK

SEQ ID NO: 43 - protein, 12 aa, Artificial Sequence
  SYNTHESIZED
ABAKABKAKABG

SEQ ID NO: 44 - protein, 10 aa, Artificial Sequence
  SYNTHESIZED
AGAGKGAGAG

SEQ ID NO: 45 - protein, 10 aa, Artificial Sequence
  SYNTHESIZED
AGAGEGAGAG

SEQ ID NO: 46 - protein, 4 aa, Artificial Sequence
  SYNTHESIZED
SPKR

SEQ ID NO: 47 - protein, 4 aa, Artificial Sequence
  SYNTHESIZED
SPKA

SEQ ID NO: 48 - protein, 4 aa, Artificial Sequence
  SYNTHESIZED
SPAR

SEQ ID NO: 49 - protein, 4 aa, Artificial Sequence
  SYNTHESIZED
SAKR

SEQ ID NO: 50 - protein, 4 aa, Artificial Sequence
  SYNTHESIZED
SGKR

SEQ ID NO: 51 - protein, 4 aa, Artificial Sequence
  SYNTHESIZED
APKR

SEQ ID NO: 52 - protein, 5 aa, Artificial Sequence
  SYNTHESIZED
KAAAK

SEQ ID NO: 53 - protein, 6 aa, Artificial Sequence
  SYNTHESIZED
KAAAAK

SEQ ID NO: 54 - protein, 9 aa, Artificial Sequence
  SYNTHESIZED
GGGGSGGGG

SEQ ID NO: 55 - protein, 4 aa, Artificial Sequence
  SYNTHESIZED
RAAR

SEQ ID NO: 56 - protein, 4 aa, Artificial Sequence
  SYNTHESIZED
RAAR

SEQ ID NO: 57 - protein, 5 aa, Artificial Sequence
  SYNTHESIZED
RAAAR

SEQ ID NO: 58 - protein, 6 aa, Artificial Sequence
  SYNTHESIZED
RAAAAR

SEQ ID NO: 59 - protein, 7 aa, Artificial Sequence
  SYNTHESIZED
KLFSSKQ

SEQ ID NO: 60 - protein, 4 aa, Artificial Sequence
  SYNTHESIZED
RVRR

SEQ ID NO: 61 - protein, 6 aa, Artificial Sequence
  SYNTHESIZED
KGGGGK

SEQ ID NO: 62 - protein, 4 aa, Artificial Sequence
  SYNTHESIZED
AAKK

SEQ ID NO: 63 - protein, 4 aa, Artificial Sequence
  SYNTHESIZED
LAKK

SEQ ID NO: 64 - protein, 4 aa, Artificial Sequence
  SYNTHESIZED
FAKK

SEQ ID NO: 65 - protein, 4 aa, Artificial Sequence
  SYNTHESIZED
ALKK

SEQ ID NO: 66 - protein, 4 aa, Artificial Sequence
  SYNTHESIZED
LLKK

SEQ ID NO: 67 - protein, 4 aa, Artificial Sequence
  SYNTHESIZED
FLKK

SEQ ID NO: 68 - protein, 4 aa, Artificial Sequence
  SYNTHESIZED
AFKK

SEQ ID NO: 69 - protein, 4 aa, Artificial Sequence
  SYNTHESIZED
LFKK

SEQ ID NO: 70 - protein, 4 aa, Artificial Sequence
  SYNTHESIZED
FFKK

SEQ ID NO: 71 - protein, 4 aa, Artificial Sequence
  SYNTHESIZED
AKKK

SEQ ID NO: 72 - protein, 4 aa, Artificial Sequence
  SYNTHESIZED
AKKK

SEQ ID NO: 73 - protein, 4 aa, Artificial Sequence
  SYNTHESIZED
FKKK

SEQ ID NO: 74 - protein, 4 aa, Artificial Sequence
  SYNTHESIZED
AKAK

SEQ ID NO: 75 - protein, 4 aa, Artificial Sequence
  SYNTHESIZED
LKAK

SEQ ID NO: 76 - protein, 4 aa, Artificial Sequence
  SYNTHESIZED
ARRK

SEQ ID NO: 77 - protein, 4 aa, Artificial Sequence
  SYNTHESIZED
RARK

SEQ ID NO: 78 - protein, 4 aa, Artificial Sequence
  SYNTHESIZED
AAQK

SEQ ID NO: 79 - protein, 4 aa, Artificial Sequence
  SYNTHESIZED
RAQK

SEQ ID NO: 80 - protein, 4 aa, Artificial Sequence
  SYNTHESIZED
ARQK

SEQ ID NO: 81 - protein, 4 aa, Artificial Sequence
  SYNTHESIZED
LLQK

SEQ ID NO: 82 - protein, 4 aa, Artificial Sequence
  SYNTHESIZED
FFQK

SEQ ID NO: 83 - protein, 4 aa, Artificial Sequence
  SYNTHESIZED
LKRR

SEQ ID NO: 84 - protein, 4 aa, Artificial Sequence
  SYNTHESIZED
GLKR

SEQ ID NO: 85 - protein, 4 aa, Artificial Sequence
  SYNTHESIZED
QRKR

SEQ ID NO: 86 - protein, 4 aa, Artificial Sequence
  SYNTHESIZED
NKKR

SEQ ID NO: 87 - protein, 5 aa, Artificial Sequence
  SYNTHESIZED
AKAKG

SEQ ID NO: 88 - protein, 5 aa, Artificial Sequence
  SYNTHESIZED
LKAKG

SEQ ID NO: 89 - protein, 5 aa, Artificial Sequence
  SYNTHESIZED
AKAKS

SEQ ID NO: 90 - protein, 5 aa, Artificial Sequence
  SYNTHESIZED
LKAKS

(19) United States
(12) Patent Application Publication
(10) Pub. No.: US 2022/0017572 A1
(43) Pub. Date: Jan. 20, 2022

(54) GEMINOID LIPOPEPTIDE COMPOUNDS AND THEIR USES

(71) Applicants: Erasmus University Medical Center Rotterdam, Rotterdam (NL); Stichting Katholieke Universiteit, Nijmegen (NL)

(72) Inventors: Bob Johan SCHOLTE, Rotterdam (NL); Martinus Christiaan FEITERS, Nijmegen (NL); Mark DAMEN, Nijmegen (NL)

(21) Appl. No.: 17/374,437

(22) Filed: Jul. 13, 2021

Related U.S. Application Data
(63) Continuation of application No. 16/310,343, filed on Dec. 14, 2018, now abandoned.

(30) Foreign Application Priority Data
Jun. 17, 2016 (NL) 2016987

(51) Int. Cl. C07K 5/103; C07K 5/083; A61P 31/14; A61K 38/08; C07K 5/09; C07K 7/06; C07K 5/072; C07K 7/08; A61K 38/05; C07K 5/113; A61K 38/07; A61K 38/00

(52) U.S. Cl. CPC C07K 5/101; Y02A 50/30; A61P 31/14; C07K 5/1008; A61K 38/08; C07K 5/0806; C07K 5/0817; C07K 7/06; C07K 5/06104; C07K 7/08; A61K 38/05; C07K 5/1021; A61K 38/07; A61K 38/00; C07K 5/0808

(57) ABSTRACT

Disclosed are geminoid peptide-like compound according to Formula I:

R¹—C(═O)—Zₙ—NR³-R² (I)

in which R¹ and R² are each independently saturated, partly saturated or unsaturated, straight, branched or cyclic alkyl chains, wherein R¹ has a number of C atoms of 11 or more, preferably 11 to 19, and R² has a number of C atoms of 12 or more, preferably 12 to 20; R³ is hydrogen or C₁-C₆ alkyl; n is an integer from 1-15;
each Z independently is an amino acid residue, wherein Zₙ comprises an N-terminus attached to C(═O) and a C-terminus that is attached to NR³, for use as a medicament.

### CROSS-REFERENCE TO RELATED APPLICATIONS

This application claims the benefit of U.S. application Ser. No. 16/310,343, filed Dec. 14, 2018, which is a national stage application of International Application Number PCT/NL2017/050403, which was filed on Jun. 16, 2017, which claims priority to Netherlands Application Number 2016987 filed on Jun. 17, 2016, each of the disclosures of which are herein incorporated by reference in their entirety.

### FIELD OF THE INVENTION

The invention is in the field biochemistry and pharmaceuticals, more specifically pertaining to a structural chemical platform for providing peptide-like compounds for use as a medicament. Particularly, the invention relates to protease inhibitors which are useful for therapy, especially to therapy in relation to viral infections.

### REFERENCE TO A SEQUENCE LISTING

This application contains a Sequence Listing that has been submitted in ASCII format via EFS-Web and is hereby incorporated by reference in its entirety. The ASCII copy, created on Oct. 2, 2019, is named Untitled ST25.txt, and is 15.39 KB bytes in size.

### BACKGROUND

Proteases are involved in many metabolic or catabolic reactions in the cell. Hence, they are also involved or deemed to be involved in pathological processes, especially when they become active at times or places where they are not supposed to become active. Proteases are currently classified into six broad groups:
Serine proteases
Threonine proteases
Cysteine proteases
Aspartate proteases
Metalloproteases
Glutamic acid proteases

The threonine and glutamic-acid proteases were not described until 1995 and 2004, respectively. The mechanism used to cleave a peptide bond involves making an amino acid residue in which the serine, cysteine, and threonine (proteases) or a water molecule (aspartic acid, metallo- and glutamic acid proteases) are nucleophilic so that it can attack the peptide carboxyl group. One way to make a nucleophile is by a catalytic triad, where a combination of a histidine with an aspartic acid residue is used to activate serine, cysteine, or threonine as a nucleophile.

Within each of the broad groups the proteases have been classified, by Rawlings and Barrett, into families of related proteases. For example within the serine proteases families are labelled Sx where S denotes the serine catalytic type and the x denotes the number of the family, for example S1 (chymotrypsins). An up to date classification of proteases into families is found in the MEROPS database (merops.sanger.ac.uk).

Protease reactions often occur in cascades where a compound is made active by deleting a part of it, which then acts as a protease for activating a second protein, and so on. The classical example of such a pathway is the blood coagulation cascade that ends with the conversion of fibrinogen into fibrin. It will be clear that any activation of such a protease cascade at a time or place where it is not needed will be dangerous to health.

For this and other reasons, some researchers have addressed the ‘degradome’ which is defined as the complete set of proteases present in an organism (Quesada, V. et al., 2009, Nucl. Acids Res. 37:D239-D243). Results of studies on these degradomes have resulted in a database on a website (degradome.uniovi.es) which website also provides detailed information about generic diseases of proteolysis. An overview of mammalian and more specifically human proteases that are involved in diseases and which thus would serve as a target for pharmaceutical protease inhibitors is given in Table A. This list is not complete, but serves to illustrate the extremely wide scope of the field

TABLE A
Mammalian proteases that are involved in diseases (Data obtained
from degradome.uniovi.es). The reference under the heading OMIM ® refers
to the Online Mendelian Inheritance in Man ® database developed by staff of
the John Hopkins Institute and hosted by NCBI

| Protease | Gene | Locus | Disease | OMIM |
|---|---|---|---|---|
| angiotensin | ACE | 17q23 | Renal tubular | 106180 |
| converting enzyme | | | dysgenesis | |
| aminoacylase 1 | ACY1 | 3p21 | Aminoacylase 1 | 104620 |
| | | | deficiency | |
| ADAM9 | ADAM9 | 8p11 | Cone-rod dystrophy | 612775 |
| ADAM10 | ADAM10 | 15q21 | Late-onset Alzheimer's | 602192 |
| | | | disease | |
| ADAM17 | ADAM17 | 2p25 | Inflammatory Skin | 603639 |
| | | | and Bowel Disease, | |
| | | | carcinoma | |
| ADAMTS-10 | ADAMTS10 | 19p13 | Weill-Marchesani syndrome | 277600 |
| ADAMTS-13 | ADAMTS13 | 9q34 | Thrombotic thombocytopenic | 274150 |
| | | | purpura | |
| ADAMTS-17 | ADAMTS17 | 15q26 | Weill-Marchesani syndrome | 277600 |
| ADAMTS-18 | ADAMTS18 | 16q23 | Knobloch syndrome | 267750 |
| procollagen I N- | ADAMT | 5q23 | Ehlers-Danlos | 225410 |
| endopeptidase | S2 | | syndrome type VIIC | |
| Afg3-like protein 2 | AFG3L2 | 18p11 | Dominant hereditary | 610246 |
| | | | ataxia SCA28 | |
| Afg3-like protein 2 | AFG3L2 | 18p11 | Spastic Ataxia- | 604581 |
| | | | Neuropathy Syndrome | |
| glycosylasparaginase | AGA | 4q34 | Aspartylglucosaminuria | 208400 |
| aspartoacylase (np) | ASPA | 17p13 | Canavan disease | 271900 |
| complement | BF | 6p21 | Atypical hemolytic | 235400 |
| factor B | | | uremic syndrome | |
| procollagen C- | BMP1 | 8p21 | Osteogenesis | 259420 |
| proteinase | | | imperfecta, type III | |
| complement | C1R | 12p13 | C1r deficiency | 216950 |
| component C1r | | | | |
| complement | C1S | 12p13 | C1s deficiency | 120580 |
| component | C1s | | | |
| complement | C2 | 6p21 | C2 deficiency | 217000 |
| component 2 | | | | |
| calpain 10 | CAPN10 | 2q37 | Autosomal recessive | 605286 |
| | | | intellectual disability | |
| | | | (ARID) | |
| calpain 3 | CAPN3 | 15q15 | Limb-girdle muscular | 253600 |
| | | | dystrophy type 2A | |
| caspase-10 | CASP10 | 2q33 | Autoimmune | 603909 |
| | | | lymphoproliferative | |
| | | | syndrome (II) | |
| caspase-2 | CASP2 | 11q22 | Autosomal recessive | 600639 |
| | | | intellectual disability(ARID) | |
| caspase-8 | CASP8 | 2q33 | Autoimmune | 601859 |
| | | | lymphoproliferative | |
| | | | syndrome (I) | |
| tripeptidyl-peptidase I | CLN2 | 11p15 | Neuronal ceroid lipofuscinosis | 204500 |
| carboxypeptidase A6 | CPA6 | 8q13 | Duane retraction syndrome | 126800 |
| carboxypeptidase E | CPE | 4q33 | Hyperproinsulinemia | 125853 |
| | | | and diabetes | |
| carboxypeptidase N | CPN1 | 10q24 | Carboxypeptidase N | 212070 |
| | | | deficiency | |
| chymotrypsin C | CTRC | 1p36 | Hereditary pancreatitis | 167800 |
| cathepsin C | CTSC | 11q14 | Papillon-Lefevre and | 245000 |
| | | | Haim-Munk syndromes | |
| cathepsin D | CTSD | 11p15 | Neuronal ceroid lipofuscinosis | 610127 |
| cathepsin K | CTSK | 1q21 | Pycnodysostosis | 265800 |
| cylindromatosis protein | CYLD1 | 16q12 | Cylindromatosis | 132700 |
| complement factor D | DF | 19p13 | DF deficiency | 134350 |
| desert hedgehog protein | DHH | 12q13 | Partial gonadal dysgenesis | 607080 |
| DJ-1 (putative protease) | DJ-1 | 1p36 | Parkinson disease type VII | 606324 |
| dihydropyrimidinase (np) | DPYS | 8q22 | Dihydropyrimidinase | 222748 |
| | | | deficiency | |
| endothelin-converting | ECE1 | 1p36 | Hirschprung disease | 142623 |
| enzyme 1 | | | | |
| neutrophilelastase | ELA2 | 19p13 | Cyclic neutropenia | 162800 |
| | | | cystic fibrosis COPD, Asthma | |
| coagulation factor Xa | F10 | 13q34 | Factor X deficiency | 227600 |
| coagulation factor Xia | F11 | 4q35 | Factor XI deficiency | 264900 |
| coagulation factor XIIa | F12 | 5q35 | Factor XII deficiency | 234000 |
| coagulation factor XIIa | F12 | 5q35 | Hereditary | 610619 |
| | | | angioedema type III | |
| thrombin | F2 | 11p11 | Hyperprothrombinemia/ | 176930 |
| | | | hypoprothombinemia | |
| coagulation factor VIIa | F7 | 13q34 | Factor VIIa deficiency | 227500 |
| coagulation factor Ixa | F9 | Xq27 | Hemophilia B | 306900 |
| FACE1/ZMPSTE24 | FACE1 | 1p34 | Progeria, | 248370 |
| | | | Mandibuloacral dysplasia | |
| gamma- | GGT1 | 22q11 | Gamma-glutamyltransferase | 231950 |
| glutamyltransferase 1 | | | deficiency | |
| haptoglobin-1 (np) | HP | 16q22 | Anhaptoglobinemia | 140100 |
| osteoblast serine protease | HTRA1 | 10q26 | CARASIL | 600142 |
| Omi/HtrA2/PRSS25 | HTRA2 | 2p12 | Parkinson disease | 168600 |
| complement factor I | IF | 4q25 | CFI deficiency | 217030 |
| indian hedgehog protein | IHH | 2q35 | Brachydactyly type A1 | 112500 |
| mitoch. inner membrane | IMMP2L | 7q31 | Gilles de la Tourette | 137580 |
| protease 2 | | | syndrome | |
| Kell blood-group protein | KEL | 7q35 | Kell blood group antigen | 110900 |
| kallikrein 4 | KLK4 | 19q13 | Hypomaturation | 204700 |
| | | | amelogenesis imperfecta | |
| plasma kallikrein | KLKB1 | 4q35 | Prekallikrein deficiency | 229000 |
| mannan-binding | MASP1 | 3q29 | 3MC | ?term = 3 |
| serine protease 1 | | | | MC |
| mannan-binding | MASP2 | 1p36 | MASP2 deficiency | 605102 |
| serine protease 2 | | | | |
| S2P protease | MBTPS2 | Xp22 | Ichthyosis follicularis, atrichia, | 308205 |
| | | | and photophobia syndrome | |
| | | | (IFAP) | |
| ataxin 3 | MJD1 | 14q32 | Machado-Joseph disease | 109150 |
| neprilysin | MME | 3q26 | Fetomaternal alloimmunisation | 120520 |
| collagenase 3 | MMP13 | 11q22 | Spondyloepimetaphyse | 602111 |
| | | | al dysplasia | |
| gelatinase A | MMP2 | 16q13 | Multicentric osteolysis | 605156 |
| | | | with arthritis | |
| gelatinase B | MMP9 | 20q13 | Metaphyseal anadysplasia, | 613073 |
| | | | chronic inflammatory lung | |
| | | | disease | |
| enamelysin | MMP20 | 11q22 | Amelogenesis imperfecta | 301200 |
| matriptase | MTSP1 | 11q24 | Ichthyosis with hypotrichosis | 606797 |
| nasal embryonic | NELFnp | 9q34 | Kallmann syndrome | 608137 |
| LHRH factor | | | | |
| presenilins-assoc. | PARL | 3q27 | Parkinson's disease | 168600 |
| rhomboid like | | | | |
| proprotein convertase 1 | PCSK1 | 5q15 | Obesity | 600955 |
| proprotein convertase 5 | PCSK5 | 9q21 | VACTERL/Caudal regression/ | 192350 |
| | | | Currarino syndrome-like | |
| proprotein convertase 9 | PCSK9 | 1p32 | Hyperlipoproteinemia type III | 144400 |
| X-Pro dipeptidase | PEPD | 19q13 | Prolidase deficiency | 170100 |
| PHEX endopeptidase | PHEX | Xp22 | X-linked hypophosphatemia | 307800 |
| plasmin | PLG | 6q26 | Thrombophilia and | 173350 |
| | | | ligneous conjunctivitis | |
| protease, serine, 56 | PRSS56 | 2q37 | Microphtalmia, Isolated 6 | 613517 |
| lysosomal | PPGB | 20q13 | Galactosialidosis | 256540 |
| carboxypeptidase A | | | | |
| prolylendopeptidase-like | PREPL | 2p21 | Hypotonia-cystinuria syndrome | 606407 |
| protein C | PROC | 2q21 | Thrombophilia | 176860 |
| cationic trypsin | PRSS1 | 7q35 | Hereditary pancreatitis/ | 167800 |
| | | | trypsin deficiency | |
| neurotrypsin | PRSS12 | 4q28 | Nonsyndromic mental | 249500 |
| | | | retardation | |
| enteropeptidase | PRSS7 | 21q21 | Enteropeptidase deficiency | 226200 |
| presenilin 1 | PSEN1 | 14q24 | Alzheimer type 3 | 104311 |
| presenilin 1 | PSEN1 | 14q24 | Familial Acne Inversa | 142690 |
| presenilin 2 | PSEN2 | 1q42 | Alzheimer type 4 | 600759 |
| proteasome | PSMB8 | 6p21 | auto-inflammatory syndrome | 613732 |
| catalytic subunit 3i | | | | |
| proteasome | PSMB8 | 6p21 | Nakajo-Nishimura syndrome | 256040 |
| catalytic subunit 3i | | | | |
| reelin | RELN | 7q22 | Lissencephaly syndrome | 257320 |
| renin | REN | 1q32 | Renal tubular dysgenesis | 179820 |
| rhomboid 5 homolog 2 | RHBDF2 | 17q25 | Tylosis with esophageal cancer | 148500 |
| sonic hedgehog protein | SHH | 7q36 | Holoprosencephaly type 3 | 142945 |
| paraplegin | SPG7 | 16q24 | Spastic paraplegia | 607259 |
| transferrin receptor 2 | TFR2 | 7q22 | Hemochromatosis type 3 | 604250 |
| protein (np) | | | | |
| transmembrane | TMPRSS3 | 21q22 | Deafness | 605316 |
| protease, serine 3 | | | | |
| matriptase 2 | TMPRSS6 | 22q12 | Iron-refractory iron | 609862 |
| | | | deficiency anemia | |
| ubiquitin C-terminal | UCHL1 | 4p14 | Parkinson disease type V | 191342 |
| hydrolase 1 | | | | |
| USP26 | USP26 | Xq26 | Sertoli cell-only syndrome | 305700 |
| USP9Y | USP9Y | Yq11 | Azoospermia and | 415000 |
| | | | hypospermatogenesis | |
| aminopeptidase | XPNPEP3 | 22q13 | Nephronophthisis-like | ncbi.nlm. |
| P homologue | | | nephropathy | nih.gov |

It is submitted that finding molecules that safely inhibit proteases would be of benefit for treating diseases such as mentioned in table A.

Further, it is well documented that tumor progression, i.e. proliferation, migration, invasion and metastasis is dependent on regulatory proteases on several levels. This includes intracellular maturation of proteins, such as furin, or turnover such as proteasomes, secreted metalloproteinases (MMP) involved in extracellular matrix turnover, as well as membrane bound proteases (ADAM) involved in regulation of growth factors.

Therefore antagonists of such proteases aimed to inhibit cancer progression are desired pharmaceutical compounds.

As can be derived from Table A proteases of the same categories expressed by epithelial mesenchymal and myeloid cells play a role in the resolution of inflammation and tissue repair. Therefore, protease inhibitors targeting such enzymes as MMP, ADAM can be of use in chronic inflammatory disease such as cystic fibrosis (CF), asthma, COPD, rheumatoid arthritis, Crohn's disease and other chronic inflammatory diseases.

For example, ADAM17 may be seen as a key regulator of several pathways involved in CF lung pathology, which makes it a potential therapeutic target for CF and related chronic lung disease (FIG. 1). Several studies support the concept that ADAM17 is a potential target for experimental therapeutics. Major fields of application are tumour progression and chronic inflammation (Crohn's disease, rheumatoid arthritis, multiple sclerosis) [1, 2]. ADAM dependent signaling is considered a valid target in the treatment of tumor progression, affecting epithelial proliferation migration and differentiation [3].

In the past decade, significant progress was made towards the development of specific ADAM inhibitors with therapeutic potential, and a wide variety of experimental compounds with efficacy in the nanomolar range and acceptable bioavailability and toxicity in animal studies are available [4, 5]. Several clinical applications are under investigation. However, none of the compounds has been approved for phase III studies yet.

The application of such compounds is associated with several caveats. Since the active sites of the ADAM and MMP metalloproteinases are highly related, the specificity of the inhibitors is not absolute. Side effects may occur through inhibition of other than the intended pathway. Conversely, closely related ADAMS, such as ADAM10 and ADAM17, have different but overlapping target protein target spectra [1]. This suggests that functional redundancy should be expected, and absolute selectivity might actually be a disadvantage in clinical application of inhibitors.

However, not only mammalian proteases are important in this respect. A further particular example of proteases involved in pathogenic processes are viral proteases, which are the enzymes used by viruses to cleave nascent proteins for final assembly of new virions.

In a number of infective diseases, such as those caused by the Flaviviridae family of pathogenic viruses (Dengue, West Nile, Hepatitis C), the viral protein has to be split (FIG. 2, [Leung et al., 2001]) in structural and non-structural proteins by the concomitant action of viral and host proteases for it to become infective after expression by the host cell. A host protease involved is furin (proprotein convertase) which also plays physiological roles such as conversion of the proinsulin to insulin. Furin is a serine protease for which crystal structures are available [Henrich et al., 2003; Wheatley & Holyoak, 2007].

Because of the ever increasing threat of viral infections it is desirable to have good inhibitors for the proteases, but in view of their similar substrate preferences it is difficult to design inhibitors that interact preferentially with viral proteases instead of host proteases.

(viral) Proteases not only play a role in pathological processes. Proteases are also not desired in production processes for proteins. Such processes can take place in mammalian or other cell cultures, but also in bacterial cultures. In the latter case it can happen that such bacterial cultures are infected with bacterial phages that also use proteases in their life cycle. Thus, application of protease inhibitors in the field of protein production processes also is desired.

Therefore there is a need for new small molecule protease inhibitors. Generally, however, application of such inhibitors is limited due to a lack of specificity towards the members of the enzyme families, which have different but overlapping expression patterns and physiological functions. Further, systemic delivery of such molecules is often prohibitive due to toxic and off-target effects, whereas targeted delivery is not feasible.

Also, it would be desired to provide a chemical platform which would provide a structural set-up for making peptide-like compounds that can be delivered to cells, and wherein the peptide structure can be tailored so as to accommodate use in treating a set of different protease-mediated diseases.

Further, it would be desired to provide peptide-like compounds suitable for use in the treatment of Dengue.

Chemically-modified peptides are known for various uses. A background reference is WO 01/98362 which describes antimicrobial peptides having a sequence of two to seven amino acids, wherein both the carboxyl terminus and the amino terminus are suggested to be modified with a great variety of side-chains. The antimicrobial use is described with reference to a wide range of application areas, viz. for inhibition and termination of microbial growth, particularly bacterial growth, for industrial, pharmaceutical, household, and personal care use. The reference does not address protease inhibition.

A background reference related to a protease is GB 1 564 317. Herein dipeptide derivatives are disclosed, wherein the amino terminal side is substituted with an aromatic, aliphatic or cycloaliphatic group up to six carbon atoms (viz. phenyl, substituted phenyl, lower cyclo-alkyl, or n-(C₁-C₆-alkyl. The carboxyl terminal side is substituted with an amide, sulfonyl amide or sulfinyl amide group. These compounds are said to be suitable for use in the treatment of degenerative diseases associated with the action of elastase-like enzymes.

A further reference related to inhibitors of the enzyme elastase, is U.S. Pat. No. 4,528,133. Disclosed are tripeptide and tetrapeptide alkylamides, which have a short alkyl side chain on the amino terminal side, and an alkylcarbonylamino group with 2 to 12 carbon atoms, an alkenyl with 6 to 12 carbon atoms, or a benzyloxycarbonylamino group on the carboxyl terminal side.

Another background reference is WO 2008/137758. Therein modified amino acids or peptides having 2-20 amino acids are disclosed, wherein either or both of the carboxyl and amino termini have a lipophilic tail. The disclosed compounds serve as enhancing agents for the delivery of various drugs, typically nucleic acid molecules to be delivered to cells. In WO 2008/137758 the aforementioned compounds are not used as drug substances.

Another background reference is WO 2009/046220, which relates to lipopeptides for delivery of nucleic acids. Compounds are referred to that comprise a peptide having 2 to 100 amino acid residues, and having a lipophilic group attached to at least one terminus of the peptide, or at least one amino acid residue of the peptide. The peptides are disclosed for a use as an excipient in the delivery of nucleic acids, whereby the nucleic acid is a therapeutically active substance, and the peptide is a formulation aid.

Yet another teaching of a similar use as in the foregoing references, is provided by Damen, M. et al. (J. Contr. Release, 145:33-39, 2010). Therein gemini-like amphiphilic peptides are disclosed for use as vectors for transport of polynucleotides into cells. Here too, the peptides are a formulation aid, with the polynucleotides serving as therapeutically active substances.

A further background reference is Tomohiro Hikima et al., International Journal of Pharmaceutics, Vol. 443, 2013, 288-292, which relates to the gemini surfactant sodium dilauramidoglutamine lysine. This surfactant is investigated as a chemical enhancer for the skin penetration of L-ascorbic acid 2-glucoside. It is not itself used as a drug substance. The disclosed compound does not have a conventional peptide bond. Lysine is connected to one glutamate by its alpha-amino group, and to another glutamate by its epsilon-amino group.

Ten Brink et al., J. Pept.Sci, 2006, 12, 686-692 presents a protocol to label the C-terminus of a peptide with a moiety that is functionalized with a primary amine. Several of such modified peptides are exemplified. The reference foresees a use of the C-modified peptides in click chemistry, biological assays, in making noncovalent stabilized peptides and giant amphiphiles, or as functional building blocks.

The variety of teachings in the art does not allow meeting the present desires. Some of these teachings are too general in nature to provide guidance specifically towards antiviral compounds, let alone to protease inhibition. Others are too specific in nature to provide the desired versatility to create a to provide a structural chemical platform, and other teachings turn into a direction of a non-therapeutic use of modified peptides, viz. as an excipient or formulation aid.

### SUMMARY OF THE INVENTION

In order to better address one or more of the foregoing desires, the invention, in one aspect, provides geminoid peptide-like compounds according to Formula I:

R¹—C(═O)—Zₙ—NR³-R² (I)

in which R¹ and R² are each independently saturated, partly saturated or unsaturated, straight, branched or cyclic alkyl chains, wherein R¹ has a number of C atoms of 11 or more, preferably 11 to 19, and R² has a number of C atoms of 12 or more, preferably 12 to 20; R³ is hydrogen or C₁-C₆ alkyl; n is an integer from 1-15;
each Z independently is an amino acid residue, wherein Zₙ comprises an N-terminus attached to C(═O) and a C-terminus that is attached to NR³, for use as a medicament.

In another aspect, the invention presents a pharmaceutical composition comprising, as the sole drug substance, a geminoid peptide-like compound according to Formula I, for use as a medicine.

In another aspect, the invention presents novel geminoid peptide-like compounds according to Formula I as defined above, wherein R¹ comprises 11 to 13 carbon atoms, R² comprises 12 to 14 carbon atoms, and n is 4.

In a still further aspect, the invention presents novel geminoid peptide-like compounds according to Formula I as defined above, wherein R¹ comprises 13 carbon atoms and R² comprises 14 carbon atoms.

In yet another aspect, the invention concerns novel geminoid peptide-like compounds according to Formula I as defined above, with the proviso that said compound is not any of the compounds:

(SEQ ID NO: 39)
C₁₁H₂₃CO-GANPNAAG-NH—C₁₈H₃₇;
(SEQ ID NO: 39)
C₁₃H₂₇CO-GANPNAAG-NH—C₁₈H₃₇;
(SEQ ID NO: 39)
C₁₅H₃₁CO-GANPNAAG-NH—C₁₈H₃₇;
(SEQ ID NO: 39)
C₁₇H₃₅CO-GANPNAAG-NH—C₁₈H₃₇;
(SEQ ID NO: 39)
C₁₅H₃₁CO-GANPNAAG-NH—C₁₆H₃₃;
(SEQ ID NO: 39)
C₁₃H₂₇CO-GANPNAAG-NH—C₁₄H₂₉;
(SEQ ID NO: 39)
C₁₁H₂₃CO-GANPNAAG-NH—C₁₂H₂₅;
(SEQ ID NO: 40)
C₁₅H₃₁CO-KGGGK-NH—C₁₆H₃₃;
(SEQ ID NO: 41)
C₁₅H₃₁CO-KGGK-NH—C₁₆H₃₃;
C₁₅H₃₁CO-KGK-NH—C₁₆H₃₃;
C₁₅H₃₁CO-KK-NH—C₁₆H₃₃;
(SEQ ID NO: 41)
C₁₇H₃₃CO-KGGK-NH—C₁₈H₃₅;
C₁₇H₃₃CO-KGK-NH—C₁₈H₃₅;
(SEQ ID NO: 42)
C₁₇H₃₃CO-KAAK-NH—C₁₈H₃₅;
(SEQ ID NO: 43)
C₁₅H₃₁CO-ABAKABKAKABG-NH—C₁₆H₃₃;
(SEQ ID NO: 44)
C₁₇H₃₅CO-AGAGKGAGAG-NH—C₁₈H₃₇;
(SEQ ID NO: 45)
C₁₇H₃₅CO-AGAGEGAGAG-NH—C₁₈H₃₇;
(SEQ ID NO: 46)
C₁₇H₃₅CO-SPKR-NH—C₁₈H₃₇;
(SEQ ID NO: 47)
C₁₇H₃₃CO-SPKA-NH—C₁₈H₃₅;
(SEQ ID NO: 48)
C₁₇H₃₃CO-SPAR-NH—C₁₈H₃₅;
(SEQ ID NO: 49)
C₁₇H₃₃CO-SAKR-NH—C₁₈H₃₅;
(SEQ ID NO: 50)
C₁₇H₃₃CO-SGKR-NH—C₁₈H₃₅;
(SEQ ID NO: 51)
C₁₇H₃₃CO-APKR-NH—C₁₈H₃₅;
(SEQ ID NO: 46)
C₁₇H₃₃CO-SPKR-NH C₁₈H₃₅;
wherein C₁₇H₃₃CO- stands for oleoyl, and
C₁₈H₃₅ stands for oleyl.

General Remark

In the following description, the compounds of formula (I) are sometimes defined with reference to a shorthand notation, whereby the H atoms in R¹ and R², the C(═O) group attached to R¹ and the NR³ group attached to R², are not shown. This refers to a notation Cₚ—Zₙ—C_{q}, whereby Zn has the aforementioned meaning, and p and q are integers such that Cₚ indicates the number of carbon atoms in R¹—C(═O), and C_{q} indicates the number of carbon atoms in R². Hereby the left-hand side of the molecule as described is R¹ and the right-hand side is R². E.g., the notation C₁₆-KAAAK-C₁₆ (SEQ ID NO:52) implies R¹=n-C₁₅H₃₁, R²=n-C₁₆H₃₃; the C═O group linking R¹ to the left-hand K is not shown; R³ is H and the NH group linking the right-hand K to R² is not shown.

### DESCRIPTION OF THE DRAWINGS

FIG. 1 depicts a schematic representation of the proposed role of ADAM17 in CF lung disease

FIG. 2 shows flavivirus polyprotein (shaded: NS2B co-factor and proteolytic domain of NS3) processing by host proteases (black arrows, bottom) and the virus-encoded protease complex NS2B.NS3 (open arrows above) [adapted from Leung et al., 2001].

FIG. 3A shows binding of a substrate with amino acids numbered P1, P2, etc. from the site of cleavage (indicated with towards the N-terminus, and P′₁, P′₂, towards the C-terminus, with a protease active site with complimentary binding sites S1, S2, etc. and S′₁, S′₂. FIG. 3B shows FRET peptide substrate for the protease assay with a fluorescent N-terminal Abz (aminobenzoyl) group (λ_{exc} 320 nm, λₑₘ 420 nm) which is quenched by the C-terminal EDDnp (ethylenediamine-dinitrofluorophenyl) group before the peptide is cleaved. FIG. 3C shows the general structure of a methyl coumarin amide Rₙ-XR-MCA (Ac, Rₙ=Me; Bz, Rₙ=Ph; Z, Rₙ=BnO) substrate, which gives a fluorescent product (λ_{exc} 380 nm, λₑₘ 460 nm) upon hydrolysis at the position indicated by the arrow.

FIG. 4A shows inhibition of furin by C₁₆-K(G)ₙK-C₁₆ (A, n=2); FIG. 4B shows inhibition of furin by C₁₆-K(G)ₙK-C₁₆(B, n=3); FIG. 4C shows inhibition of furin by C₁₆-K(G)ₙK-C₁₆ (C, n=4).

FIG. 5A, FIG. 5B and FIG. 5C show Dengue NS3-NS2B peptidase hydrolysis of Abz-AKRRSQ-EDDnp (SEQ ID NO:1) in 50 mM Tris-HCl pH 9.0. FIG. 5A Inhibitors: (◯) C₁₆-KAAK-C₁₆ (SEQ ID NO:42), (▪)C₁₆-KAK-C₁₆, (▴)C₁₆-KGGK-C₁₆(SEQ ID NO:41). FIG. 5B Substrates: (◯) Abz AKRRSQ-EDDnp (SEQ ID NO:1) and (●) Suc AAPF-MCA with different concentrations of C₁₆-KGGK-C₁₆ (SEQ ID NO:41). FIG. 5C Followed in time at different C₁₆-KGGK-C₁₆ (SEQ ID NO:41) concentrations.

FIG. 6A and FIG. 6B show Dengue NS3-NS2B peptidase hydrolysis of Abz AKRRSQ-EDDnp (SEQ ID NO:1) in the presence of C₁₆-KK-C₁₆ (KR), C₁₆-KAK-C₁₆ (KAK), C₁₆-KAAK-C₁₆ (KAAK; (SEQ ID NO:42)), C₁₆-KAAAAK-C₁₆ (KAAAAK; (SEQ ID NO:53)), C₁₆-KGK-C₁₆ (KOK), C₁₆-KGGK-C₁₆ (KOOK; (SEQ ID NO:41)), and C₁₆-KGGGK-C₁₆ (KGGGK; (SEQ ID NO:40)). “◯” means enzyme activity without inhibitor. FIG. 6A E+K means that the inhibitor+enzyme were pre-incubated for 0, 15 and 30 min and then the substrate was added. FIG. 6B K means that the inhibitor was pre-incubated for 0, 15 and 30 min and then the enzyme and substrate were added.

FIG. 7 shows Vero cells treated with different peptides of the C₁₆-Z-C₁₆ type. Vero Cells, which are used to test Dengue virus infection and replication, were grown to confluence, and treated for 48 hrs with different concentrations of C16-Z-C16 geminoids, as indicated in the figure. Upper left: carrier alone (DMSO), upper right: C16-RR-C16, 6 uM, insert 3 uM. Middle left: C16 KAAK-C16 (SEQ ID NO:42) 25 uM, middle right: 12 uM. Lower left: C16 KAK-C16 25 uM lower right 12 uM. At high concentrations, geminoid treated cells show accumulation of intracellular vesicles, consistent with inhibition of the intracellular processing protease furin. The C16 KAK-C16 treated cells were less affected by this than C16-KAAK-C16 (SEQ ID NO:42) treated cells.

FIG. 8 shows inhibition of Dengue virus replication in VERO cells by C16-KAK-C16, infection at different multiplicity of infection.

1×10⁶ Vero E6 cells were plated per well into a 6-wells plate and incubated overnight at 37° C. Next day, the DENV-2/NGC virus stock was diluted to 10⁴ TICD₅₀/ml, 10³ TICD₅₀/ml, 10² TICD₅₀/ml and 1 ml of the respective virus dilutions was added to each well. Wells contained approximately 80% confluent monolayers of Vero cells. After an incubation period of 1 hour at 37° C., cells were washed twice with medium (DMEM) and medium containing 2% methyl cellulose was added to the wells. To this medium C16-KAK-C16 10 uM was added (W) in DMSO, or an equivalent amount of DMSO (0.05%) was added (W/O). Plate was incubated at 37° C. for two days. Methyl cellulose overlays were removed and cells were fixed with absolute ethanol. Cells were subsequently incubated with specific DENY monoclonal antibody for 1 hour at 37° C., followed by incubation with HRPO-labeled rabbit-anti mouse conjugate. Positive plaques were counted after incubation with AEC substrate chromogen.

FIG. 9 shows inhibition of denguevirus replication in VERO cells by different concentrations of C16-KAK-C16.

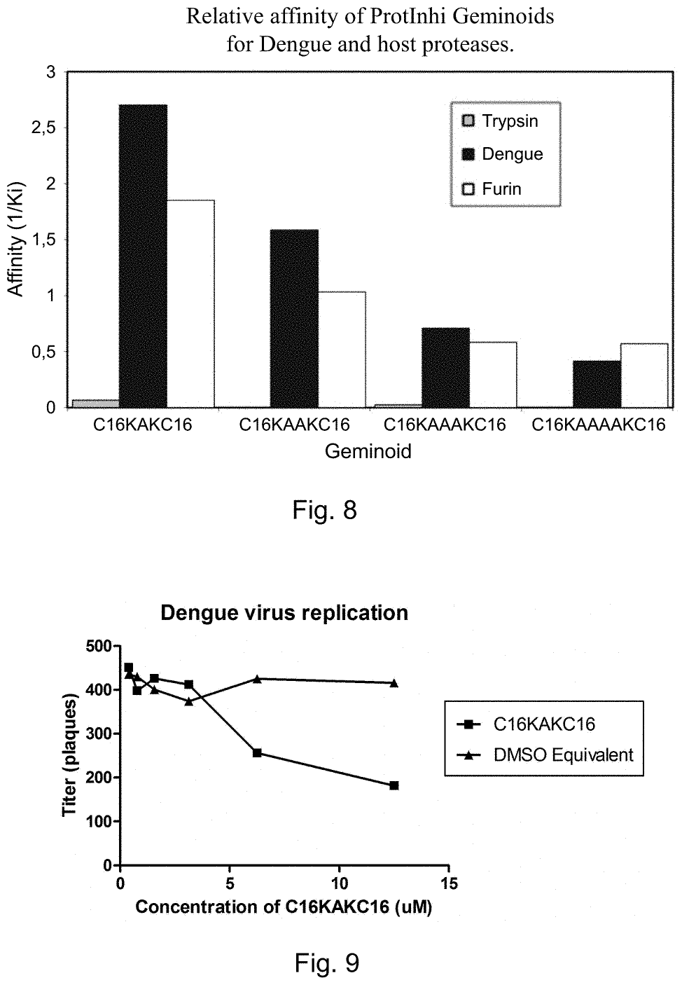

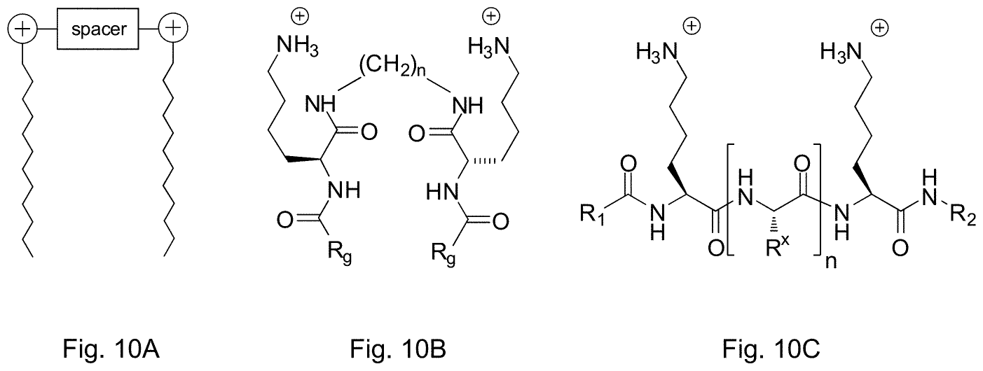

The experiment was performed as described in the legend of FIG. 8 at a viral dose of 10³ TICD₅₀/ml in all plates. Different concentrations of C16-KAK-C16, or an equivalent amount of DMSO were applied as indicated in the figure.

FIG. 10A, FIG. 10B and FIG. 10C show general structures of Gemini compounds. FIG. 10A, general structure of (cationic) gemini surfactants [Menger & Keiper, 2000]; FIG. 10B, cationic gemini surfactant R_{g}-n-R_{g} (R_{g}, alkyl tail; n, number of methylene groups in spacer) based on lysine [Kirby et al., 2003]; FIG. 10C, gemini-like alkylated peptide (geminoid) R₁-Lys-[AA]ₙ-Lys-R₂, where R₁ and R₂ are alkyl tails and R^{x} are the side chains of n Ala or Gly [ten Brink et al., 2006; Damen et al., 2010].

### DETAILED DESCRIPTION OF THE INVENTION

In a broad sense, the invention is based on the judicious insight to provide geminoid peptides, having hydrocarbon side chains at both the carbonyl and the amino terminus of the peptide, with a number of carbon atoms of at least 12. It has been found that this allows providing useful antiviral geminoid peptides, particularly being protease inhibitors.

It is thereby emphasized that the invention relates to a medical use of geminoid peptides that hitherto have not been known for such use. Rather, several background references on geminoid peptides relate to a use as a formulation aid. The invention particularly presents a composition for use as a medicine (i.e., a pharmaceutical composition) comprising a geminoid peptide-like compound according to Formula (I) as defined above, and in all of its embodiments described hereinbefore and hereinafter, as the sole drug substance.

In this application the term ‘geminoids’ or ‘gemini-like peptides’ or ‘bi(s)-alkylated peptide’ or ‘BAPS’ is used for those compounds that have a number of amino acids connected through a peptide binding, wherein the C-terminal and the N-terminal peptide both are provided with an alkyl chain. The general synthesis and properties of BAPs has been described in ten Brink et al. (2006) and Damen et al. (2010).

These compounds have the general formula (I):

R¹—C(═O)—Zₙ—NR³-R² (I)

in which R¹ and R² are each independently saturated, partly saturated or unsaturated, straight, branched or cyclic alkyl chains with a number of C atoms of 12 or more, preferably 12 to 20; R³ is hydrogen or C₁-C₆ alkyl; n is an integer from 1-15;
each Z independently is an amino acid residue, wherein Zₙ comprises an N-terminus attached to C(═O) and a C-terminus that is attached to NR³.

Preferably Z is —NR³—C(R⁴R⁵)—C(═O)—, in which R₄ is selected from side chains occurring in natural amino acids and R₅ is selected from the group consisting of hydrogen, C₁-C₆ straight or branched, saturated, partly saturated or unsaturated alkyl, and alkoxy.

Each Z independently preferably is an amino acid selected from the group consisting of natural amino acids, beta-alanine (bAla), 4-aminomethyl phenylalanine (Amf), 4-guanidine phenylalanine (Gnf), 4-aminomethyl-N-isopropyl phenylalanine (Iaf), 3-pyridyl alanine (Pya), 4-piperidyl alanine (Ppa), 4-aminomethyl cyclohexyl alanine (Ama), 4-aminocyclohexyl alanine (Aca), ornithine (Orn), citrulline, hydroxylysine (Hyl), allo-hydroxylysine (aHyl), 6-N-methyllysine (MeLys), desmosine (Des), isodesmosine (Ide), 2-aminoadipic acid (Aad), 3-aminoadipic acid (bAad), 2-aminobutyric acid (Abu), 4-aminobutyric acid (4Abu), 6-aminohexanoic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), 2,2′-diaminopimelic acid (Dpm), 2-3-diaminopropionic acid (Dpr), N-ethylglycine (EtGly), N-ethylasparagine (EtAsn), 3-hydroxyproline (3Hyp), 4-hydroxyproline (4Hyp), allo-isoleucine (AIle), sarcosine (MeGly), N-methylisoleucine (MeIle), N-methylvaline (MeVal), norvaline (Nva), and norleucine (Nle). Preferably each Z is independently a natural amino acid.

Preferably n is an integer from 1-10, and more preferably from 3-8, more preferably from 3-7, more preferably from 3-6, and more preferably from 3-5.

As an example the structure of such a compound is given below, the compound R¹—C(═O)—KZₙK—NH—R², wherein a left (N-terminal) and a right (C-terminal) Z amino acid is provided by a lysine residue, which can be connected via a further number of amino acids.

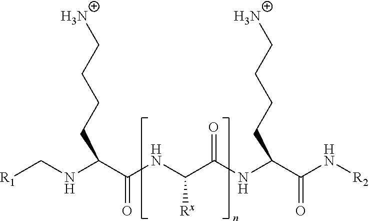

Furthermore, in this application the notation C₁₆-KAAAK-C₁₆ (SEQ ID NO:52) implies R₁=n-C₁₅H₃₁, R₂=n-C₁₆H₃₃; the C═O group linking R₁ to K is not shown; R³ is H and the NH group thus linking K to R² is not shown); Zₙ is represented by the sequence KAAAK (Lys-Ala-Ala-Ala-Lys; (SEQ ID NO:52)). Note that due to the presence of the linker C═O, the number of C atoms at the right and the left of the amino acid sequence is 16. Thus, the short hand notation reads C₁₆-KAAAK-C₁₆ (SEQ ID NO:52).

In one aspect, the invention is directed to the compounds of formula (I) for use as a medicament. Particularly, this use is as a medicament in the treatment of viral infection. Accordingly, the invention also pertains to a method of treatment of a viral infection, by the administration, to a subject in need thereof, an effective amount of a compound according to the above-identified formula (I).

In part, the invention relates to novel compounds. In one aspect, these compounds are characterized by satisfying the above formula I, wherein the number of carbon atoms in R¹—C(═O) and R² is 14. In another aspect, these compounds are characterized by satisfying the above formula I, wherein the number of carbon atoms in R¹—C(═O) and R²,each independently, is 12 to 14, and n is 4. The foregoing compounds are believed to provide an optimum in terms of combined properties such as a viral inhibitory effect and ease of formulation.

In an alternative embodiment, the novel compounds are those satisfying the above formula I, with the proviso that said compound is not any of the following compounds:

(SEQ ID NO: 39)
C₁₁H₂₃CO-GANPNAAG-NH-C₁₈H₃₇;
(SEQ ID NO: 39)
C₁₃H₂₇CO-GANPNAAG-NH-C₁₈H₃₇;
(SEQ ID NO: 39)
C₁₅H₃₁CO-GANPNAAG-NH-C₁₈H₃₇;
(SEQ ID NO: 39)
C₁₇H₃₅CO-GANPNAAG-NH-C₁₈H₃₇;
(SEQ ID NO: 39)
C₁₅H₃₁CO-GANPNAAG-NH-C₁₆H₃₃;
(SEQ ID NO: 39)
C₁₃H₂₇CO-GANPNAAG-NH-C₁₄H₂₉;
(SEQ ID NO: 39)
C₁₁H₂₃CO-GANPNAAG-NH-C₁₂H₂₅;
(SEQ ID NO: 40)
C₁₅H₃₁CO-KGGGK-NH-C₁₆H₃₃;
(SEQ ID NO: 41)
C₁₅H₃₁CO-KGGK-NH-C₁₆H₃₃;
C₁₅H₃₁CO-KGK-NH-C₁₆H₃₃;
C₁₅H₃₁CO-KK-NH-C₁₆H₃₃;
(SEQ ID NO: 41)
C₁₇H₃₃CO-KOOK-NH-C₁₈H₃₅;
C₁₇H₃₃CO-KGK-NH-C₁₈H₃₅;
(SEQ ID NO: 42)
C₁₇H₃₃CO-KAAK-NH-C₁₈H₃₅;
(SEQ ID NO: 43)
C₁₅H₃₁CO-ABAKABKAKABG-NH-C₁₆H₃₃;
(SEQ ID NO: 44)
C₁₇H₃₅CO-AGAGKGAGAG-NH-C₁₈H₃₇;
(SEQ ID NO: 45)
C₁₇H₃₅CO-AGAGEGAGAG-NH-C₁₈H₃₇;
(SEQ ID NO: 46)
C₁₇H₃₅CO-SPKR-NH-C₁₈H₃₇;
(SEQ ID NO: 47)
C₁₇H₃₃CO-SPKA-NH-C₁₈H₃₅;
(SEQ ID NO: 48)
C₁₇H₃₃CO-SPAR-NH-C₁₈H₃₅;
(SEQ ID NO: 49)
C₁₇H₃₃CO-SAKR-NH-C₁₈H₃₅;
(SEQ ID NO: 50)
C₁₇H₃₃CO-SGKR-NH-C₁₈H₃₅;
(SEQ ID NO: 51)
C₁₇H₃₃CO-APKR-NH-C₁₈H₃₅;
(SEQ ID NO: 46)
C₁₇H₃₃CO-SPKR-NH-C₁₈H₃₅;
wherein C₁₇H₃₃CO- stands for oleoyl,
and C₁₈H₃₅ stands for oleyl.

The compounds herein excluded are those which have been incidentally disclosed by Damen et al. or ten Brink et al. The skilled person will understand that the capital letters refer to an internationally accepted way of indicating amino acids. A list of natural amino acids, with their general abbreviations is given in Table 1.

TABLE 1

| | Three-Letter | Single -Letter |
|---|---|---|
| Amino Acid | Abbreviation | Abbreviation |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Z is an amino acid residue. The term “residue” is hereby used to indicate that both the carboxyl and the amino groups of each Z are bound, either to another Z, or to the C(═O) or —NR³ groups shown in Formula I. More specifically, in the above-mentioned geminoid peptide-like compounds Z is based on an amino acid chosen from the group of natural amino acids, beta-alanine (bAla), 4-aminomethyl phenylalanine (Amf), 4-guanidine phenylalanine (Gnf), 4-aminomethyl-N-isopropyl phenylalanine (Iaf), 3-pyridyl alanine (Pya), 4-piperidyl alanine (Ppa), 4-aminomethyl cyclohexyl alanine (Ama), 4-aminocyclohexyl alanine (Aca), ornithine (Orn), citrulline, hydroxylysine (Hyl), allo-hydroxylysine (aHyl), 6-N-methyllysine (MeLys), desmosine (Des), isodesmosine (Ide), 2-aminoadipic acid (Aad), 3-aminoadipic acid (bAad), 2-aminobutyric acid (Abu), 4-aminobutyric acid (4Abu), 6-aminohexanoic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), 2,2′-diaminopimelic acid (Dpm), 2-3-diaminopropionic acid (Dpr), N-ethylglycine (EtGly), N-ethylasparagine (EtAsn), 3-hydroxyproline (3Hyp), 4-hydroxyproline (4Hyp), allo-isoleucine (AIle), sarcosine (Me Gly), N-methylisoleucine (MeIle), N-methylvaline (MeVal), norvaline (Nva), and norleucine (Nle). Also preferred are geminoid peptide like compounds wherein n is an integer from 1-10 and more preferably from 3-8, more preferably from 3-7, more preferably from 3-6 more preferably from 3-5. Further preferred are geminoid peptide-like compounds wherein NR³ is NH. Further preference is expressed for geminoid peptide-like compounds wherein Z is a natural amino acid. It is also preferred to use geminoid peptide-like compounds wherein the alkyl chains are partly saturated.

Further preferred are geminoid peptide-like compounds wherein Zₙ is a part of the molecule that is capable of binding to a protease recognition site on a substrate, preferably wherein said protease recognition site is chosen from the group of recognition sites specified in Tables B and C, AKRRSQ (SEQ ID NO:1), RₘXR, in which m is an integer of 2 or higher and X is any amino acid, SPLAQAVKSSSRK (SEQ ID NO:2), GSDMELPLPRNITEGEARGSVILTVKPIFEEF (SEQ ID NO:3) and GSKTEEISEVNLDAEFRHDS (SEQ ID NO:4).

In an interesting embodiment of the various applicable aspects of the invention as broadly described above, R¹—C(═O) and R² in the compounds of formula (I), each independently, have a number of carbon atoms of at least 14, preferably at least 16. Preferably, the number of carbon atoms for the groups R¹—C(═O) and R², each independently, is 24 or lower, such as 22, 20, 18, 16, 14, or 12. Preferably, the number of carbon atoms for the groups R¹—C(═O) and R², each independently, is 12 to 19, more preferably 13 to 18, more preferably 15 to 17.

In another interesting embodiment, either or both of R¹ and R² are straight chain hydrocarbons, preferably mono-unsaturated. In yet another embodiment, either or both of R¹ and R² are branched chain hydrocarbons, preferably saturated.

Preferably, the integer n in the compounds of formula (I) is 4 or 8, most preferably 4.

In an interesting embodiment, Zₙ in the compounds of Formula (I) is devoid of proline in the second position. In another interesting embodiment, proline is absent.

It is noted that, in accordance with conventional peptide nomenclature, the peptide sequence is numbered from the N-terminal side to the C-terminal side of the peptide.

In a further interesting embodiment, serine is not present in a position at the N-terminal side of an arginine or a lysine (i.e., in conventional numbering, a serine is not present before an arginine or a lysine). In a still further interesting embodiment, a serine is present and at least one argine or lysine, wherein the serine is positioned at the C-terminal side of the argine or lysine.

In a further interesting embodiment, Zₙ in the compounds of Formula (I) has a hydrophobic amino acid in the first position. Natural hydrophobic amino acids are glycine, alanine, valine, leucine, isoleucine, proline, phenylalanine, methionine, and tryptophan. Preferred hydrophobic amino acids are leucine and phenylalanine.

Further preferred are geminoid peptide-like compounds having the general formula:

C₁₅C(═O)—KA_{q}K—NH—C₁₆ (II)

with q being an integer of from 1 to 15, preferably 1 to 7, more preferably from 1 to 5, more preferably from 1 to 4, more preferably from 1 to 3, and more preferably 1 or 2, and wherein C₁₅ is a saturated, partly saturated or unsaturated straight, branched or cyclic alkyl chain of 15 carbon atoms and C₁₆ is a saturated, partly saturated or unsaturated straight, branched or cyclic alkyl chain of 16 carbon atoms.

The therapeutic use of said geminoid peptide-like compounds is, for instance, in treating protease mediated disease. The therapeutic use is preferably in antiviral therapy, in inflammation and in ADAM17 mediated diseases, such as ulcerative colitis, rheumatoid arthritis, cystic fibrosis, COPD, IPF, Crohn's disease, multiple sclerosis and atherosclerosis. If the use is in antiviral therapy, preferably said antiviral therapy is therapy against Flaviviridae, more preferably therapy against dengue.

Further part of the invention are non-therapeutic uses of a geminoid peptide-like compound, having a general formula according to Formula I as defined above as protease inhibitors, as anti-septics, particularly for the disinfection of surfaces, and as anti-microbial agents in cell-culturing.

Salts and solvates. It may be convenient or desirable to prepare, purify, and/or handle a corresponding salt of a geminoid, as described herein, for example, a pharmaceutically acceptable salt. Examples of pharmaceutically acceptable salts are discussed in Berge et al., 1977, “Pharmaceutically Acceptable Salts,” J. Pharm. Sci., Vol. 66, pp. 1-19. Examples of suitable salts include: those derived from the following inorganic acids (such as hydrochloric, hydrobromic, hydroiodic, sulfuric, sulfurous, nitric, nitrous, phosphoric, and phosphorous acid); those derived from organic acids (such as 2-acetyoxybenzoic, acetic, ascorbic, aspartic, benzoic, camp horsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric acid); those derived from polymeric acids (such as tannic acid and carboxymethyl cellulose). Unless otherwise specified, a reference to a geminoid or geminoids also includes salt forms thereof.

It may be convenient or desirable to prepare, purify, and/or handle a corresponding solvate of a geminoid. The term “solvate” is used herein in the conventional sense to refer to a complex of solute (e.g., geminoid, salt of geminoid) and solvent. If the solvent is water, the solvate may be conveniently referred to as a hydrate, for example, a mono-hydrate, a di-hydrate, a tri-hydrate, etc. Unless otherwise specified, a reference to geminoid also includes solvate forms thereof.

One aspect of the invention pertains to a pharmaceutical composition comprising a geminoid according to Formula I or a salt or solvate thereof.

A further aspect of the invention pertains to a pharmaceutical composition comprising a geminoid according to Formula I or a salt or solvate thereof, and a pharmaceutically acceptable carrier, diluent, or excipient. Examples of suitable pharmaceutically acceptable carriers, diluents, and excipients are described below.

Unique characteristics of Geminoids We submit, based on evidence presented below that the compounds according to the above described general formula can be used as specific protease inhibitors in various clinical and non-clinical contexts.

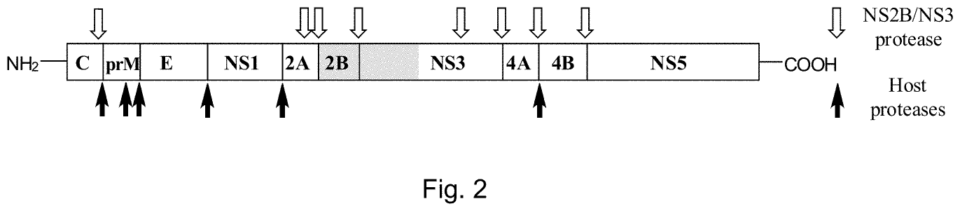

One of the main advantages of the present compounds of the invention is the specificity that is offered by the peptide sequence Zₙ, which can be optimized to be identical to or a derivative of a sequence that is capable of being targeted to an actual protease cleavage site on a substrate. Thus, preferably, the compounds of the invention comprise a moiety Z that provides for a chemical structure, preferably an amino acid structure, that is targeted to a specific protease active domain. A further major advantage of the present compounds is formed by the nanoparticle aggregation of the compounds and their adaptable interaction with cellular membranes. These two properties together offer unique opportunities for functional targeting tissue specificity and sub-cellular delivery. It is submitted that the interaction with cellular membranes and nanoparticle formation has already been described by Damen et al. (J. Controlled Release 145: 33-39, 2010), which information, especially the synthesis of the compounds as described in paragraphs 2.2 and 2.3 of the scientific document and the results depicted in FIG. 2 therein, is included herein by reference.

One aspect of the present invention pertains to the use of a geminoid according to Formula I or a salt or solvate thereof as an anti-protease agent, also indicated as protease inhibitor. In general the term ‘protease inhibitor’ relates to a compound that inhibits a protease. Many proteases are highly specific, acting on single or small families of substrates, but many single substrates can also be cleaved by several proteases. For many proteases the actual amino acid sequence that acts as the substrate is known. These substrate sequences often are short sequences (maximizing 4-8 amino acids). As is shown in the experimental part, the substrate sequence, or a derivative thereof can be designed to form the main core of the geminoid compound (the part Zₙ of the general formula). In such a way a compound can be constructed that is ideally suited to bind with a single protease.

In a number of infective diseases, such as those caused by the Flaviviridae family of pathogenic viruses (Dengue, West Nile, Hepatitis C), the viral protein has to be split (FIG. 2, [Leung et al., 2001]) in structural and non-structural proteins by the concomitant action of viral and host proteases for it to become infective after expression by the host cell. A host protease involved is furin (proprotein convertase) which also plays essential physiological roles such as conversion of the proinsulin to insulin. Furin is a serine protease for which crystal structures are available [Henrich et al., 2003; Wheatley & Holyoak, 2007].

The active site of the dengue protease is in the N-terminal part of NS3 which is also a serine protease with catalytic triad Asp79-His51-Ser135, but requires NS2A (CF40) for activity; the inhibition reported here was studied on a NS3-NS2A construct (CF40-GGGGSGGGG-NS3; (SEQ ID NO:54)) which has also been structurally characterized [Erbel et al., 2006; Luo et al. 2008].

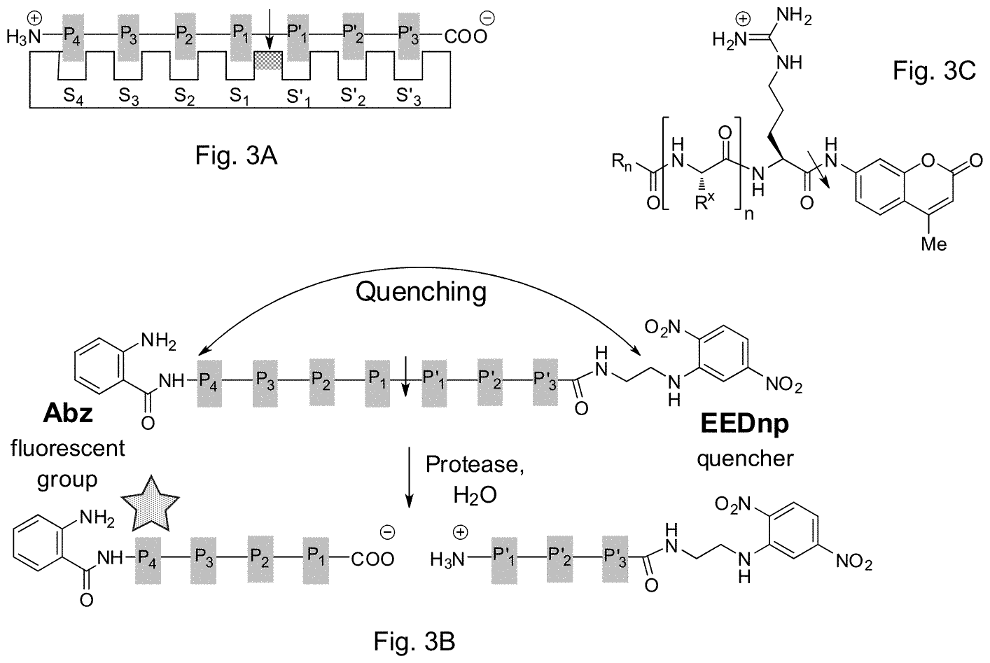

The substrate specificity of proteases can be studied with FRET substrates of the Abz-EEDnp type, where the fluorescence of the N-terminal Abz (aminobenzoyl) group is quenched by the C-terminal EDDnp (ethylenediamine-dinitrofluorophenyl) group until the peptide is split (FIG. 3B). These studies have shown that the preferred substrates for furin have the general structure —R^{P4}-X^{P3}-(K/R)^{P2}-R^{P1}↓X^{P1′}-X^{P2′}-X^{P3′}-X^{P4′} [Izidoro et al., 2009], while the best substrate for dengue protease is Abz-AKRR↓SQ-EDDnp (SEQ ID NO:1) [Gouvea et al., 2007]; this means that the ideal furin and dengue protease substrates have cationic residues in positions P₁-P₂-P₄ and P₁-P₂-P₃, respectively, next to the site of cleavage (↓) in the direction of the N-terminus. For inhibition studies, the 7-amino-4-methyl coumarin amide (MCA) derivatives of general structure Rₙ-XR-MCA ([Melo et al., 2001] (FIG. 3C) are used.

For ADAM17 a highly susceptible recognition site is formed by SPLAQA∧VKSSSRK (SEQ ID NO:2), the aggrecanase recognition sequence from aggrecan is GSDMELPLPRNITEGE∧ARGSVILTVKPIFEEF (SEQ ID NO:3), and the BACE recognition sequence from β-amyloid precursor protein is GSKTEEISEVNL∧DAEFRHDS (SEQ ID NO:4) (the ∧ indicates the protease cleavage site).

Further specific recognition sites and cleavage sites for some serine proteases are given in the below table B.

TABLE B
Target sequences for serine proteases and splicing site.

| Serine protease | Bond split upon activation | SEQ ID NO: |
|---|---|---|
| Prothrombin | Glu-Gly-Arg [symbol] Ile-Val-Glu-Gly | 5 |
| Pro-urokinase | Arg-Phe-Lys [symbol] Ile-Ile-Gly-Gly | 6 |
| trypsinogen | Asp-Asp-Lys [symbol] Ile-Val-Gly-Gly | 7 |
| chymotrypsinogen | Leu-Ser-Arg Ile-Val-Asn-Gly | 8 |
| Pro-elastase | Val-Tyr-Arg [symbol] Val-Val-Gly-Glu | 9 |
| Pro-subtilisin | Ala-Gly-Lys [symbol] Ser-Asn-Gly-Glu | 10 |
| Coagulation factor V | Gly-Ile-Arg [symbol] Ser-Phe-Arg-Phe | 11 |
| Coagulation factor VII | Pro-Gln-Arg [symbol] Ile-Val-Gly-Gly | 12 |
| Coagulation factor IX | Asp-Phe-Thr-Arg [symbol] Val-Val-Gly-Gly | 13 |
| Coagulation factor X | Asn-Leu-Thr-Arg [symbol] Ile-Val-Gly-Gly | 14 |
| Coagulation factor XII | Ser-Met-Thr-Arg [symbol] Val-Val-Gly-Gly | 15 |
| Coagulation factor XI | Ile-Lys-Pro-Arg [symbol] Ile-Val-Gly-Gly | 16 |
| Kallikrein | Thr-Ser-Thr-Arg [symbol] Ile-Val-Gly-Gly | 17 |
| Plasminogen | Pro-Gly-Arg [symbol] Val-Val-Gly-Gly | 18 |
| Cathepsin G | Ala-Gly-Glu [symbol] Ile-Ile-Gly-Gly | 19 |

Sequences obtained from SWISS-PROT, GenBank or PIR databases.

Substrate cleavage sites for various caspases are given in the below table C.

TABLE C
Substrate cleavage sites of proteases of the caspase family.
Preferred sequences

| | | | | | | | | |
|---|---|---|---|---|---|---|---|---|
| Caspase-1 | YEVD | [symbol] | WEHD | [symbol] | LEVD | [symbol] | WVAD | [symbol] |
| | (SEQ ID NO: 20) | | (SEQ ID NO: 21) | | (SEQ ID NO: 22) | | (SEQ ID NO: 23) | |
| Caspase-2 | VDVAD | [symbol] | DEHD | [symbol] | LDESD | [symbol] | | |
| | (SEQ ID NO: 24) | | (SEQ ID NO: 25) | | (SEQ ID NO: 26) | | | |
| Caspase-3 | IETD | [symbol] | DMQC | [symbol] | | | | |
| | (SEQ ID NO: 27) | | (SEQ ID NO: 28) | | | | | |
| Caspase-4 | LEVD | [symbol] | WEHD | [symbol] | LEHD | [symbol] | WVAD | [symbol] |
| | (SEQ ID NO: 22) | | (SEQ ID NO: 21) | | (SEQ ID NO: 29) | | (SEQ ID NO: 23) | |
| Caspase-5 | WEHD | [symbol] | LEHD | [symbol] | LEAD | [symbol] | | |
| | (SEQ ID NO: 21) | | (SEQ ID NO: 29) | | (SEQ ID NO: 30) | | | |
| Caspase-6 | VEID | [symbol] | VEHD | [symbol] | VKMD | [symbol] | VNLD | [symbol] |
| | (SEQ ID NO: 31) | | (SEQ ID NO: 32) | | (SEQ ID NO: 33) | | (SEQ ID NO: 34) | |
| Caspase-7 | DEVD | [symbol] | | | | | | |
| | (SEQ ID NO: 35) | | | | | | | |
| Caspase-8 | IETD | [symbol] | LETD | [symbol] | | | | |
| | (SEQ ID NO: 27) | | (SEQ ID NO: 36) | | | | | |
| Caspase-9 | LEHD | [symbol] | VEHD | [symbol] | | | | |
| | (SEQ ID NO: 29) | | (SEQ ID NO: 32) | | | | | |
| Caspase-10 | IEAD | [symbol] | AEVD | [symbol] | VEHD | [symbol] | | |
| | (SEQ ID NO: 37) | | (SEQ ID NO: 38) | | (SEQ ID NO: 32) | | | |

There is thus a large variation in specific sites that can be used for constructing the protease inhibitors according to the invention. The sequences as given above can be used, but also sequences that are derived from these sequences, i.e. by adding, deleting or substituting one or more of the amino acids. Substitutions can take the form of natural amino acids, but also the non-natural amino acids as listed above may be used. An example is the 1,2,3-triazole moiety that can be obtained by the Cu-catalysed so-called ‘click’ reaction between an amphiphilic peptide fragment appended with an alkyne and another one with an azide. Other less reactive analogues of the amide bond are the compounds in which the carboxylic acid part of the amide has an alpha-keto group (so another, but more reactive, carbonyl next to the carbonyl involved in the covalent bond with N), or in which the amine part of the amine bond is replaced by a hydrazine (so 2 N atoms between the carbonyl and the C of the next amino acids instead of 1 as in the amide). It is also possible that amino acid like moieties according to the —NCOR⁴R⁵ schedule as defined above are inserted.

Accordingly, the invention comprises methods to inhibit proteases by using a geminoid compound according to Formula I. Such methods may be performed, for example, in vitro, as part of an assay. Such methods may also be performed, for example, in vivo, by administration of a geminoid according to Formula I or a salt or solvate thereof to a patient. Another aspect of the present invention pertains to a geminoid compound according to Formula I or a salt or solvate thereof, for use in a method of treatment of the human or animal body by therapy.

Another aspect of the present invention pertains to a geminoid compound according to Formula I or a salt or solvate thereof, for use in a method of treatment, for example, in a method of treatment or prophylaxis of (including, e.g., reducing the risk of) a disease condition as described herein. Another aspect of the present invention pertains to a geminoid compound according to Formula I or a salt or solvate thereof, for use in a method of treatment of a disease condition as described herein. Another aspect of the present invention pertains to a geminoid compound according to Formula I or a salt or solvate thereof, for use in a method of prophylaxis of (including, e.g., reducing the risk of) a disease condition as described herein.

Another aspect of the present invention pertains to use of a geminoid compound according to Formula I or a salt or solvate thereof in the manufacture of a medicament for use in a method of treatment or prophylaxis, for example, in a method of treatment or prophylaxis of (including, e.g., reducing the risk of) a disease condition as described herein.

Another aspect of the present invention pertains to use of a geminoid compound according to Formula I or a salt or solvate thereof in the manufacture of a medicament for use in a method of treatment, for example, in a method of treatment of a disease condition as described herein.

Another aspect of the present invention pertains to use of a geminoid compound according to Formula I or a salt or solvate thereof in the manufacture of a medicament for use in a method of prophylaxis, for example, in a method of prophylaxis of (including, e.g., reducing the risk of) a disease condition as described herein.

Another aspect of the present invention pertains to a method of treatment or prophylaxis, for example, a method of treatment or prophylaxis of (including, e.g., reducing the risk of) a disease condition as described herein, comprising administering to a patient in need of said treatment or prophylaxis a therapeutically- or prophylactically-effective amount of a geminoid compound according to Formula I or a salt or solvate thereof, preferably in the form of a pharmaceutical composition.

Another aspect of the present invention pertains to a method of treatment, for example, a method of treatment of a disease condition as described herein, comprising administering to a patient in need of said treatment a therapeutically-effective amount of a geminoid compound according to Formula I or a salt or solvate thereof, preferably in the form of a pharmaceutical composition.

Another aspect of the present invention pertains to a method of prophylaxis, for example, a method of prophylaxis of (including, e.g., reducing the risk of) a disease condition as described herein, comprising administering to a patient in need of said prophylaxis a prophylactically-effective amount of a geminoid compound according to Formula I or a salt or solvate thereof, preferably in the form of a pharmaceutical composition. In one embodiment, the disease condition is a disease condition that is mediated by a protease, such as a viral protease, intracellular proteases such as furin or proteasomes, extracellular metalloproteases, such as MMP, Neutrophil elastase (NE), and membrane-bound metalloproteinases including ADAMS (e.g. ADAM17, ADAM10, ADAM33), and Meprins. The term “treatment” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition.

Unless otherwise specified, treatment as a prophylactic measure (i.e., prophylaxis) is encompassed by the term “treatment”. For example, use with patients who have not yet developed the condition, but who are at risk of developing the condition, is encompassed by the term “treatment” but is more specifically described by the term “prophylaxis”. Both absolute prophylaxis and probabilistic prophylaxis are encompassed by the term “prophylaxis”. Thus, “prophylaxis” of a disease condition encompasses “reducing the risk of’ that disease condition.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. Similarly, the term “prophylactically-effective amount,” as used herein, pertains to that amount of an active compound, or a material, composition or dosage form comprising an active compound, which is effective for producing some desired prophylactic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously. For example, a geminoid compound according to Formula I or a salt or solvate thereof may also be used in combination therapies, e.g., in conjunction with other agents, for example, other anti-viral agents, antibiotic agents, anti-cancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets. For example, it may be beneficial to combine treatment with a geminoid compound according to Formula I or a salt or solvate thereof with one or more other (e.g., 1, 2, 3, 4) agents or therapies, for example, treatment with one or more of: AZT, Tamaflu®, Tofacitinib (JAR inhibitor), Velkade or related (Proteasome inhibitor).

In one embodiment, a geminoid compound according to Formula I or a salt or solvate thereof is combined with one or more (e.g., 1, 2, 3, 4) additional therapeutic agents. One aspect of the present invention pertains to a geminoid compound according to Formula I or a salt or solvate thereof, in combination with one or more additional therapeutic agents. The particular combination would be at the discretion of the physician who would select dosages using his or her common general knowledge and dosing regimens known to a skilled practitioner.

The agents (i.e., a geminoid compound according to Formula I or a salt or solvate thereof, plus one or more other agents, including one or more other geminoid compounds) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s). The agents (i.e., a geminoid compound according to Formula I or a salt or solvate thereof, plus one or more other agents, including one or more other geminoid compounds) may be formulated together in a single dosage form, or alternatively, the individual agents may be formulated separately and presented together in the form of a kit, optionally with instructions for their use, as described below.

A geminoid compound according to Formula I or a salt or solvate thereof may also be used as part of an assay, for example, an in vitro assay, for example, in order to determine whether a candidate host is likely to benefit from treatment with the compound.

A geminoid compound according to Formula I or a salt or solvate thereof may also be used as a standard or comparator, for example, in an assay, in order to identify other active compounds.

Another aspect of the present invention pertains to a kit comprising (a) a geminoid compound according to Formula I or a salt or solvate thereof, preferably provided in the form of a pharmaceutical composition and in a suitable container and/or with suitable packaging; and (b) instructions for use, for example, written instructions on how to administer the active compound.

The written instructions may also include a list of indications for which a geminoid compound according to Formula I or a salt or solvate thereof is a suitable treatment.

The geminoid compound according to Formula I or salt or solvate thereof, or the pharmaceutical composition comprising a geminoid compound according to Formula I or a salt or a solvate thereof may be administered to a subject by any convenient route of administration, whether systemically/peripherally or topically (i.e., at the site of desired action). Routes of administration include, but are not limited to, oral (e.g., by ingestion); buccal; sublingual, transdermal (including, e.g., by a patch, plaster, etc.); transmucosal (including, e.g., by a patch, plaster, etc.); intranasal (e.g., by nasal spray); ocular (e.g., by eyedrops), pulmonary (e.g., by inhalation or insufflation therapy using, e.g., via an aerosol or powder, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The subject/patient may be a chordate, a vertebrate, a mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a Iagomorph (e.g., a rabbit), avian (e.g., a bird), canine (e.g., a dog), feline (e.g., a cat), equine (e.g., a horse), porcine (e.g., a pig), ovine (e.g, a sheep), bovine (e.g., a cow), a primate, simian (e.g., a monkey or ape), a monkey (e.g., marmoset, baboon), an ape (e.g., gorilla, chimpanzee, orangutan, gibbon), or a human. Furthermore, the subject/patient may be any of its forms of development, for example, a fetus. In a preferred embodiment, the subject/patient is a human. Further, the subject can be a plant, chosen form a monocotyledonous plant, such as a grain plant or a bulbous plant, a dicotyledonous plant, a fern, a moss, or even a micro-organism, if said micro-organism suffers from viral pathogens. Accordingly also bacteria, suffering from bacteriophages, can be considered as subject for the present invention.

While it is possible for the active compound (i.e, a geminoid compound according to Formula I or a salt or solvate thereof) to be administered alone, it is preferable to present it as a pharmaceutical formulation (e.g., composition, preparation, medicament) comprising at least one active compound, as defined above, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound. The term “pharmaceutically acceptable” as used herein pertains to compounds, ingredients, materials, compositions, dosage forms, etc, which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known to the skilled person in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary. The formulation may be prepared to provide for rapid or slow release; immediate, delayed, timed, or sustained release; or a combination thereof. Formulations may suitably be in the form of tablets (including, e.g., coated tablets), granules, powders, lozenges, pastilles, capsules (including, e.g., hard and soft gelatin capsules), cachets, pills, ampoules, boluses, pessaries, suppositories, liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tinctures, gels, pastes, ointments, creams, lotions, oils, foams, sprays, mists, or aerosols.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers. Formulations may also suitably be provided in the form of a depot or reservoir.

The active compound may be dissolved in, suspended in, or admixed with one or more other pharmaceutically acceptable ingredients.

One preferred pharmaceutical formulation is when the active compound is presented in a liposome or other microparticulate which is designed to target the active compound, for example, to blood components or one or more organs. The geminoid compound according to Formula I is especially suitable for such a formulation, since it is well attached to the liposome particle due to the fatty alkyl chains.

Formulations suitable for oral administration (e g., by ingestion) include liquids, solutions (e.g., aqueous, non-aqueous), suspensions (e.g., aqueous, non-aqueous), emulsions (e.g., oil-in-water, water-in-oil), elixirs, syrups, etectuaries, tablets, granules, powders, capsules, cachets, pills, ampoules, boluses. Due to the amphiphilic character the geminoid compounds are soluble both in aqueous and non-aqueous solvents and typically suitable for emulsions.

Formulations suitable for buccal administration include mouthwashes, lozenges, pastilles, as well as patches, adhesive plasters, depots, and reservoirs. Lozenges typically comprise the active compound in a flavored basis, usually sucrose, mint and acacia or tragacanth. Pastilles typically comprise the active compound in an inert matrix, such as gelatin and glycerin, or sucrose and acacia. Mouthwashes typically comprise the active compound in a suitable liquid carrier.

Formulations suitable for sublingual administration include tablets, lozenges, pastilles, capsules, and pills. Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, lozenges, pastilles, as well as patches, adhesive plasters, depots, and reservoirs.

One particularly preferred oral delivery route is transmucosal for the upper respiratory pathways by using an aerosol.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base. Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the active compound and an oily phase, which may optionally comprise merely an emulsifier (otherwise known as an emulgent), or it may comprise a mixture of at least one emulsifier with a fat or an oil or with both a fat and an oil. A hydrophilic emulsifier may be included together with a lipophilic emulsifier which acts as a stabilizer, but in view of the amphiphilic character of the geminoids according to the invention, such additions do not seem necessary. Together, the emulsifier(s) with or without stabiliser(s) make up the so-called emulsifying wax, and the wax together with oil and/or fat make up the so-called emulsifying ointment base which forms the oily dispersed phase of the cream formulations. Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the active compound. Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluorornethane, dichloro-tetrafluoroethane, carbon dioxide, or other suitable gases.

Formulations suitable for ocular administration include eye drops wherein the active compound is dissolved or suspended in a suitable carrier, especially an aqueous solvent for the active compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema. Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection), include aqueous or non-aqueous, isotonic, pyrogen-free, sterile liquids (e g., solutions, suspensions), in which the active compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection Typically, the concentration of the active compound in the liquid is from about 1 ng/mL to about 10 mg/mL, for example from about 10 ng/mL to about 1 mg/mL The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

It will be appreciated by one of skill in the art that appropriate dosages of the active compound (i.e., a geminoid compound according to Formula I or a salt or solvate thereof), and compositions comprising the active compound, can vary from patient to patient and from targeted protease to targeted protease. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 50 pg to about 1 gram (more typically about 100 pg to about 25 mg) per kilogram body weight of the subject per day. Where the active compound is a salt or solvate, the amount administered is calculated on the basis of the parent compound and so the actual weight to be used is increased proportionately.

Next to systemic delivery also local delivery is contemplated and a further alternative may be targeted administration. Targeted administration can be achieved by binding a homing moiety that is specific for a particular site or molecule in the subject to the geminoid compound of the invention, thus providing a targeted geminoid compound. Homing moieties that target various possible targets (such as tumor tissue, nucleolar localization, etc.) are known to the skilled person and can be coupled to the geminoid compound using conventional chemical binding techniques.

The geminoid compounds according to Formula I can be used as substrate for proteases and as such can function as protease inhibitors. As such they can be tailored to different specific targets with high affinity by modeling the peptide part. The compounds are not only versatile with respect to the peptide part, but also with respect to the alkyl chains. The lipid part of the molecule ensures high delivery and target binding properties (as has been demonstrated by Damen et al., 2010).

Proteases that can be targeted are viral proteases, such as dengue protease, other flavivirus proteases, serine proteases, such as furin and kallikrein, extracellular metalloproteases, such as MMP, Neutrophil elastase (NE), and membrane-bound metalloproteinases including ADAM (e.g. ADAM17, ADAM10, ADAM33), blood proteases such as thrombin and plasmin, fecal proteases that cause pruritis and other proteases as mentioned in Table A or elsewhere in the present specification,

In principle any protease can be targeted, because proteases are characterized by their reactivity towards a very specific substrate, the specific target epitope/sequence of the protein that is cleaved by the protease. It is believed (and shown in the experimental section of the present application) that the geminoid compounds of the present invention are capable of being recognized by the protease for which they have been designed, i.e. bind to the protease, and thus act as a (competitive) inhibitor for the protease. Accordingly, the compounds of the present invention are suitable as pharmaceutically active compounds against a variety of diseases, which are caused or aggravated by proteases.

One particular important area is the interaction of the compounds of the present invention with MMP metalloproteinases and ADAM compounds.

Metalloproteinases involved in chronic inflammatory disease are endogenous secreted or membrane bound enzymes, which are involved in the resolution of inflammation, tissue injury and repair. These proteases regulate pro-inflammatory cytokines, growth factors, extracellular matrix remodeling enzymes, and dynamic cell-cell interactions required for repair and differentiation. These enzymes have been identified previously as important therapeutic targets. Specifically, chronic airway inflammation and recurrent exacerbations are hallmarks of cystic fibrosis (CF) and other chronic lung disease (COPD, IPF), of which the mechanisms are explained below. The frequent cycles of damage and repair under inflammatory stress result in a progressive and apparently irreversible tissue remodeling and loss of function.

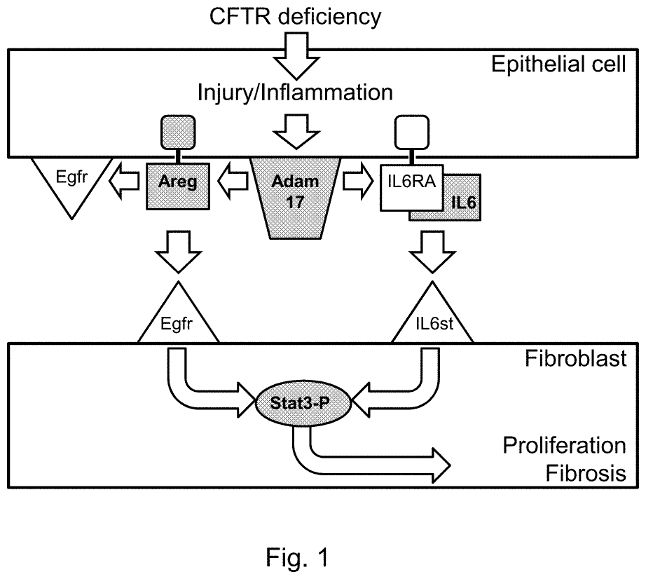

Current studies aimed at the elucidation of the signaling pathways that control tissue repair in chronic lung disease, in particular CF, have suggested that in particular ADAM17-TACE and the related ADAM10 are involved in the regulation of pro-inflammatory signaling through their substrate TNFa, and of tissue remodeling through the regulation of the EGFR and IL6RA receptors. Supported by preliminary data in a mouse model of CF and cultured bronchial epithelial cells (Scholte et al in preparation), it is proposed that a mechanism schematically depicted in FIG. 1 contributes to the development of CF lung disease. Inflammation and tissue injury triggered by CF deficiency, dramatically enhanced by colonization by opportunistic pathogens, activates EGFR and IL6 signaling. This in turn affects epithelial repair and activates subepithelial fibroblasts and smooth muscle cells. This involves at least two substrates of the epithelial ADAM17/TACE: the EGFR agonist amphiregulin and the IL6 co-receptor IL6-RA.

Other targets in this category are involved in extracellular matrix turnover and deposition, including a family of collagenases and elastases (MMP8, MMP9, neutrophil elastase), and natural inhibitors of these (TIMP, al-antitrypsin).

These enzymes have been identified as important therapeutical targets [1, 2] and many inhibitors have been developed, none of which has reached phase II clinical trial [5].

A further part of the invention is formed by the non-therapeutical use of the protease inhibitors according to the present invention. Non-therapeutic uses according to the present invention are the use of the protease inhibitors according to the present invention as research tools, e.g. for screening the presence of proteases. For such a use the geminoid protease inhibitors may be labeled, e.g. by binding to a labeling moiety or by using a radioactive moiety in the synthesis of the geminoid compound. Further, the compounds can be used in assays for specific detection of the substrate against which the geminoid compound is targeted. Also here, labeling of the compound may be applied.

A further use may be cosmetic, e.g. the use against acne or against pruritis.

Further, the protease inhibitors of the present invention may be used in food processing, as e.g. described by Garcia-Carreno, F. L. (1991, Biotechnol. Educat. 2:150-153) or be added to food or feed to increase digestibility.

Another non-therapeutic use is based on the versatility of the compounds of the invention to serve as a structural chemical platform for further screening of derivative compounds. Particularly, the compounds according to Formula I as defined above, in all its embodiments, can suitably be used in a screening method for finding further biologically active geminoid peptide-like compounds, wherein one or more compounds according to Formula I are subjected to screening in an assay for the desired activity, preferably in comparison with chemical derivatives of said one or more compounds.

According to a still further non-therapeutic use, the geminoid compounds of formula I, and the various above-described embodiments thereof, is as anti-microbial agents in cell-culturing. In yet another aspect of the invention, the geminoid compounds of formula I, and the various above-described embodiments thereof, are uses as anti-sceptics, particularly for the disinfection of surfaces, such as table surfaces, or surfaces in kitchens or bathroom, such as in households, or in public environments such as restaurants, and the like. For such a use, the compounds will generally be comprised in a carrier, typically dissolved or dispersed in an aqueous carrier, particularly in water, and can then be applied in a conventional manner, such as by spraying or via application by a cloth or other suitable article for applying a liquid disinfectant onto a surface.

### Example 1: Proteases Regulate Tissue Remodeling and Inflammation in Chronic Airway Disease

As an example of a valid application of specific and targetable geminoids in human disease we present an analysis of the role of membrane-bound proteases in the development of chronic lung disease. In chronic lung disease, activation of inflammatory responses is associated with mucus hyper secretion, epithelial metaplasia and irreversible remodelling (thickening) of the airways. Genetic factors predispose patients to excessive inflammation, tissue injury and inadequate repair. Regulatory proteases (MMP, ADAM, Meprin) play a major role in these responses. An extreme example of this is cystic fibrosis (CF), where a mutation in the CFTR chloride transport channel causes a devastating and untreatable form of chronic lung disease in more than seventy thousand patients worldwide.

Data from earlier studies in our labs show that progressive airway remodelling is observed in CT scans of CF infants, even before chronic bacterial colonisation, which is generally considered a hallmark of CF lung disease [6, 7]. While upper airway disease has traditionally received most attention in CF, it has become clear with the advent of improved imaging and lung function measurement techniques that the distal airways, in particular the membranous bronchioles are involved at a very early stage in CF [8].

The relationship between the complex pathology and the primary defect, a mutation in the CFTR chloride channel protein, is still the subject of intense investigation. The molecular mechanisms involved in progressive airway remodelling in the CF lung as discussed below yield new therapeutic approaches which can be fulfilled by the compounds of the present application.

In this context it is important to note that CF is not only a disease of the secretory epithelia. CFTR deficiency affects the behavior of alveolar macrophages [9-11] and neutrophils [12, as well as airway smooth muscle cells {Michoud et al., Am. J. Resp. Cell Molec. Biol. 40 (2009) 217, 2009]. This may independently contribute to the ‘exaggerated’ inflammatory and remodeling responses observed in patients and animal models of CF. The ‘trigger happy’ state of the immune system may contribute to the intensity of exacerbations in CF lung disease in human patients. Abnormal responses of fibroblasts and smooth muscle cells, either cell autonomous or by interaction with CF epithelial cells may determine the CF airway connective tissue pathology.

The large variation in CF lung disease progression even among patients with the same CFTR mutations, and the identification of genetic factors that influence pathology (modifier genes) further illustrates the fact that CFTR is not the only relevant therapeutic target [13].

Current efforts in the CF field are aimed at activation of the most common mutant form of CFTR, F508del (70% of all CF alleles). Although promising results were reported, none of the available compounds have shown significant long term remission in patients. Therefore, alternative approaches, including novel anti-inflammatory treatments are actively pursued [14].

The molecular mechanisms involved in the development of CF lung disease are poorly understood, despite intensive research in the past two decades. Experiments in cellular and animal models, including work in our laboratory (Scholte et al in preparation, Buijs-Offerman Thesis Erasmus MC 2011), has suggested that CFTR dysfunction affects a network of interrelated regulatory signaling molecules and their receptors, which regulates cell fate decisions. Based on our current investigations we propose that the CF deficient airways are not only challenged by recurrent infections but also respond differently to epithelial injury and inflammation.

Together, our data suggest that CF mutant mice suffer from delayed resolution of injury and inflammation, associated with enhanced activity of the EGFR and IL6 pathways. We propose that a similar mechanism is at work in the human CF lung contributing to chronic inflammation, epithelial metaplasia and connective tissue remodeling.

IL6 signalling requires the IL6R receptor IL-6RA and the co-receptor IL6 signal transducer (Gp130/IL6st). In fibroblasts and smooth muscle cells the EGFR and IL6R signals converge in activation by phosphorylation of the acute response factor STAT3, a transcription factor involved in fibrotic responses and inflammatory lung disease [15]. Recently STAT3 was recognized as an important element in progressive CF lung disease by a meta-analysis of transcriptional responses of CF compared to normal tissues [16] (FIG. 1). Similarly, IL-6R was identified as a biomarker for COPD [17, 18]

ADAMs (A Disintegrin And Metalloproteinase) form a family of ubiquitous membrane associated proteases, involved in many aspects of human development and pathology. The ADAM isoforms each interact with a different range of target proteins, many of which are involved in cell signaling, including cell adhesion proteins and receptors, cytokines and and growth factors. The canonical ADAM17/TACE, activates the pro-inflammatory factor TNFa, and is investigated as a target for inflammatory disease [2]. An ADAM17/TACE conditional (loxed) mutation in myeloid cells successfully prevented endotoxin shock in a mouse model [19]. Since CF mutant mice display a hyper inflammatory phenotype, at least in part due to abnormal behavior of alveolar macrophages [9, 20], it seems likely that inhibition of the ADAM17-TNFa pathway could attenuate this aspect of the CF phenotype.

ADAM17 is also required to activate the precursors of EGFR agonists like amphiregulin (AREG), epiregulin (EREG), heparin binding EGF (HB-EGF) and TNFα by shedding their active domain from the cell membrane, allowing autocrine and paracrine EGFR signaling [3]. EGFR activation is related to airway repair and goblet cell hyperplasia [21]. In the progression liver fibrosis amphiregulin activation of EGFR is shown to be important [22]. In experimental lung fibrosis another ADAM17 substrate and EGFR agonist TNFα plays a major role [23].

The IL6-RA receptor, which is produced by epithelial cells, is an ADAM17 substrate as well [24, 25]. This allows transactivation of IL6st (Gp130) on airway smooth muscle cells, which do not express IL6-RA, causing local VEGF release [26] (FIG. 1). IL-6 is though to contribute to fibrotic lung pathology [27].

In sum, targeting ADAM17 by an inhibitor appears an attractive approach.

In view of the many biological functions of ADAM17 in different physiological compartments [1], systemic delivery of an inhibitor is likely to have multiple and possibly adverse and contradictory effects. Further it is difficult to find small molecules that show sufficient specificity for their targets. Indeed, although many small molecule ADAM inhibitors have been developed none of these have reached Phase III. Therefore, an approach that allows targeted delivery of highly selective geminoids as is possible and contemplated in the present application would be preferable. In the case of lung diseases like CF luminal delivery by aerosol is preferred to intravenous or oral delivery

In CF and related lung disease targets are not limited to ADAM17. ADAM10 is closely related to ADAM17 and has an overlapping target spectrum. It is involved in epithelial fate decisions during tissue repair and inflammation. Also ADAM33 was recently identified as genetic determinant of Asthma and COPD [28, 29].

Meprin, another extracellular protease is also involved in CF pathology at the level of EGFR [30] and sodium channel regulation [31]; thus offering an alternative target for specific inhibition. Neutrophil elastase, which is secreted by degranulation of activated Neutrophils and causes excessive tissue damage during chronic lung inflammation is also considered to be a target of intervention.

### Example 2. Inhibition of Viral Proteases

Methods and Materials

General. Aldehyde functionalized resin (4-(4-Formyl-3-methoxyphenoxy) butyryl AM resin, loading 0.98 mmol/g) was obtained from Novabiochem and amino acids were purchased from Bachem and Novabiochem. All other chemicals were acquired from Fluka, Aldrich and Baker. The chemicals were used as received, unless stated otherwise. The polyethylene syringe barrels containing a 20 micron porous polyethylene frit were acquired from Supelco.

Mass spectra were recorded on a Thermofinnigan LCQ-ESI-ion trap. The samples were dissolved in methanol. ¹H-NMR spectra were recorded on a Bruker DMX-300 MHz at room temperature. The samples were dissolved in DMSO-d₆. In ¹H-NMR spectra, the assigned protons are in italics; s=singlet; t=triplet; qu=quintet; m=multiplet; b=a broad peak. Spectra are written in the following format: chemical shift (peak type, number of protons, subjective assignment).

Synthesis. Compounds B as depicted in FIG. 10 (Lys-based gemini surfactants) have been described before [Kirby et al., 2003] and details of the preparation and characterization have been reported elsewhere [Damen et al., Soft Matter 10 (2014) 5702-5714). The preparation of a series of alkylated peptides of type C with C₁₆-tails and Lys and Gly residues (n-C₁₅H₃₁CO-LysGlyₙLys-n-C₁₆H₃₃ or C₁₆-KGₙK-C₁₆, with 0<n<3) has been described elsewhere [ten Brink et al., 2006]. The synthesis of a number of other compounds of type C, viz. C₁₆-RAₙR-C₁₆ and C₁₂-KAₙK-C₁₂ is given below.

### Synthesis of a Series of (n-C₁₆H₃₃)-Arg-(Ala)ₙ-Arg-(n-C₁₆H₃₃).2TFA (C₁₆-KAₙK-C₁₆) for n=0-4

A reductive amination of 1.2 g aldehyde resin (1.2 mmol) as described using 2.0873 ml palmitylamine (9 mmol), 0.56 g NaCNBH₃ (9 mmol) and 550 μl AcOH in 50 ml of a 1:1 mixture of DMF/MeOH. The resin was transferred to a syringe marked (A) and one sixth of the resin was removed for experiment 8.5.4. Then, the first amino acid was coupled twice using 1.9881 g and 1.53 g Fmoc-Arg(Pmc)-OH (3.0 mmol and 2.3 mmol) 3.60 ml 1M HOBt/DMF (3.60 mmol) and 3.30 ml 1M DIPCDI/DMF (3.30 mmol). A chloranil test was negative. The resin was capped. From syringe (A), one fifth of the resin was put in a new syringe (B). To syringe (A) Fmoc-Ala-OH (592 mg, 1.5 mmol) was coupled and to syringe (B) Fmoc-Arg(Pmc))-OH (397 mg, 0.6 mmol) was coupled. Then, from syringe (A) one fourth of the resin was put in a new syringe (C). Then, Fmoc-Ala-OH (592 mg, 1.5 mmol) was coupled to it to syringe (A), Fmoc-Arg(Pmc)-OH (398 mg, 0.7 mmol) was coupled to syringe (C) and palmitic acid (154 mg, 0.6 mmol) was coupled to syringe (B). From syringe (A) one third of the resin was put in a new syringes (D). Then, Fmoc-Ala-OH (395 mg, 1.0 mmol) was coupled to it to syringe (A), Fmoc-Arg(Pmc)-OH (398 mg. 0.6 mmol) was coupled to syringe (D) and palmitic acid (154 mg, 0.6 mmol) was coupled to syringe (C). Then, from syringe (A) half of the resin was put in a new syringe (E). Then, Fmoc-Ala-OH (197 mg, 0.5 mmol) was coupled to it to syringe (A), Fmoc-Arg(Pmc)-OH (398 mg, 0.6 mmol) was coupled to syringe (E) and palmitic acid (154 mg, 0.6 mmol) was coupled to syringe (D). Next, Fmoc-Arg(Pmc)-OH (398 mg. 0.6 mmol) was coupled to syringe (A) and palmitic acid (154 mg, 0.6 mmol) was coupled to syringe (E) and finally palmitic acid (154 mg 0.6 mmol) was coupled to syringe (A). After ether washing and drying the rein in all syringes, the products were cleaved from the resin.

### n-C₁₅H₃₁C(O)-Arg-Arg-(n-C₁₆H₃₃).2TFA (C₁₆-RR-C₁₆, Syringe B)

Precipitated from ether and lyophilized from water. Yield: 49.2 mg.

LCQ-ESI Calculated (C₄₄H₈₉N₉O₃): 792.24. Found: 792.9 (19%, M+H⁺), 636.7 (6%, M-Arg+H⁺), 397.1 (100%, M+2H⁺)

### n-C₁₅H₃₁C(O)-Arg-Ala-Arg-(n-C₁₆H₃₃).2TFA (C₁₆-RAₙR-C₁₆, Syringe C)

Precipitated from ether and triturated with ether (2×). Yield: 30.2 mg.

LCQ-ESI Calculated (C₄₇H₉₄N₁₀O₄): 863.31. Found: 863.9 (25%, M+H⁺), 707.7 (8%, M-Arg+H⁺), 432.5 (100%, M+2H⁺)

### n-C₁₅H₃₁C(O)-Arg-(Ala)₂-Arg-(n-C₁₆H₃₃).2TFA (C₁₆-RA₂R-C₁₆, Syringe D; (SEQ ID NO:56)

Precipitated from ether and triturated with ether (2×). Yield: 36.6 mg.

LCQ-ESI Calculated (C₅₀H₉₉N₁₁O₅): 934.39. Found: 934.8 (25%, M+H⁺), 778.7 (8%, M-Arg+H⁺), 468.1 (100%, M+2H⁺)

### n-C₁₅H₃₁C(O)-Arg-(Ala)₃-Arg-(n-C₁₆H₃₃).2TFA (C₁₆-RA₃R-C₁₆, Syringe E; SEQ ID NO:57)

Precipitated from ether and triturated with ether (2×). Yield: 104.0 mg.

LCQ-ESI Calculated (C₅₃H₁₀₄N₁₂O₆): 1005.47. Found: 1103.6 (10%), 1005.7 (9%, M+H⁺), 920.7 (8%), 849.7 (100%, M-Arg+H⁺), 778.6 (12%), 539.5 (13%), 503.5 (100%, M+2H⁺), 468.1 (14%)

### n-C₁₅H₃₁C(O)-Arg-(la)₄-Arg-(n-C₁₆H₃₃).2TFA (C₁₆-RAₙR-C₁₆, Syringe A; SEQ ID NO:58)

Precipitated from ether and triturated with ether (2×). Yield: 192.9 mg.

LCQ-ESI Calculated (C₅₆H₁₀₉N₁₃O₇): 1076.55. Found: 1174.76%) 1076.9 (26%, M+H⁺), 920.8 (46%, M-Arg+H⁺), 539.1 (100%, M+2H⁺)

### Synthesis of a Series of (n-C₁₂H₂₅)-Lys-(Ala)ₙ-Lys-(n-C₁₂H₂₅).2TFA for n=0-4

A reductive amination of 1.2 g aldehyde resin (1.2 mmol) as described using 1.585 ml dodecyl amine (13 mmol), 754.8 mg NaCNBH₃ (12 mmol) and 680 μl AcOH in 50 ml of a 1:1 mixture of DMF/MeOH. The resin was transferred to a syringe marked (A) and the first amino acid was coupled twice using 1.7570 g and 1.7153 g Fmoc-Lys(Boc)-OH (3.7 mmol) 3.60 ml 1M HOBt/DMF (3.60 mmol) and 3.30 ml 1M DIPCDI/DMF (3.30 mmol). A chloranil test was negative. The resin was capped. From syringe (A), subsequently one sixth was removed for experiment 8.5.3 and one fifth of the resin was put in a new syringe (B). To syringe (A) Fmoc-Ala-OH (790 mg, 2.0 mmol) was coupled and to syringe (B) Fmoc-Lys(Boc)-OH (281.1 mg, 0.6 mmol) was coupled. Then, from syringe (A) one fourth of the resin was put in a new syringe (C). Then, Fmoc-Ala-OH (592 mg, 1.5 mmol) was coupled to it to syringe (A), Fmoc-Lys(Boc)-OH (281.1 mg, 0.6 mmol) was coupled to syringe (C) and lauric acid (120 mg, 0.6 mmol) was coupled to syringe (B). From syringe (A) one third of the resin was put in a new syringes (D). Then, Fmoc-Ala-OH (395 mg, 1.0 mmol) was coupled to it to syringe (A), Fmoc-Lys(Boc)-OH (281.1 mg. 0.6 mmol) was coupled to syringe (D) and lauric acid (120 mg, 0.6 mmol) was coupled to syringe (C). Then, from syringe (A) half of the resin was put in a new syringe (E). Then, Fmoc-Ala-OH (200 mg, 1.1 mmol) was coupled to it to syringe (A), Fmoc-Lys(Boc)-OH (281.1 mg. 0.6 mmol) was coupled to syringe (E) and lauric acid (120 mg, 0.6 mmol) was coupled to syringe (D). Next, Fmoc-Lys(Boc)-OH (281.1 mg. 0.6 mmol) was coupled to syringe (A) and lauric acid (120 mg, 0.6 mmol) was coupled to syringe (E) and finally lauric acid (120 mg, 0.6 mmol) was coupled to syringe (A). After ether washing and drying the rein in all syringes, the products were cleaved from the resin.

### n-C₁₁H₂₃C(O)-Lys-Lys-(n-C₁₂H₂₅).2TFA (C₁₂-KK-C₁₂, Syringe B)

No precipitation from ether. Yield: 16.0 mg.

LCQ-ESI Calculated (C₃₆H₇₃N₅O₃): 624.00. Found: 1269.7 (2M+Na⁺), 1247.6 (2M+H⁺), 624.5 (M+H⁺), 312.8 (M+2H⁺).

### n-C₁₁H₂₃C(O)-Lys-Ala-Lys-(n-C₁₂H₂₅).2TFA (C₁₂-KAK-C₁₂, Syringe C)

No precipitation from ether. Yield: 50.0 mg.

LCQ-ESI Calculated (C₃₉H₇₈N₆O₄): 695.07. Found: 1411.5 (2M+Na⁺), 1389.6 (2M+H⁺), 718.7, 695.7 (M+H⁺), 348.4 (M+2H⁺)

### n-C₁₁H₂₃C(O)-Lys-(Ala)₂-Lys-(n-C₁₂H₂₅).2TFA (C₁₂-KA₂K-C₁₂, Syringe D; (SEQ ID NO:42))

Precipitated from ether and triturated with ether (2×). Yield: 116.8 mg.

LCQ-ESI Calculated (C₄₂H₈₃N₇O₅): 766.15. Found: 1553.5 (2M+Na⁺), 1531.7 (2M+H⁺), 788.9 (M+Na⁺), 766.7 (M+H⁺), 383.9 (M+2H⁺)

### n-C₁₁H₂₃C(O)-Lys-(Ala)₃-Lys-(n-C₁₂H₂₅).2TFA (C₁₂-KA₃K-C₁₂, Syringe E; (SEQ ID NO:52))

Precipitated from ether and triturated with ether (2×). Yield: 45.6 mg.

LCQ-ESI Calculated (C₄₅H₈₈N₈O₆): 837.23. Found: 1695.6 (11%), 995.2 (11%), 859.7 (100%, M+Na⁺), 837.7 (89%, M+H⁺), 527.5 (9%), 419.3 (91%, M+2H⁺)

### n-C₁₁H₂₃C(O)-Lys-(Ala)₄-Lys-(n-C₁₂H₂₅).2TFA (C₁₂-KA₄K-C₁₂, Syringe A; (SEQ ID NO:53))

Precipitated from ether and triturated with ether (2×). Yield: 134.9 mg.

LCQ-ESI Calculated (C₄₈H₉₃N₉O₇) 908.31. Found: 1066.3 (7%, ?) 930.9 (100%, M+Na⁺), 908.8 (58%, M+H⁺), 598.5 (9%), 454.9 (52%, M+2H⁺).

Enzyme Assays

The enzyme was dissolved in 1 mL MES buffer (10 mM), 1 mM CaCl₂, pH 7.0 at 36.5° C. Substrate was added in a concentration 10 times the Kₘ, and the inhibitor compounds of type C (C₁₆-KGₙK-C₁₆, C₁₆-KAₙK-C₁₆, and C₁₆-RAₙR-C₁₆) were added in increasing concentrations (1.0, 5.0, 10.0, 20.0, 40.0 μL) from a stock solution of 2 mg in 1 mL DMSO. The residual activity was measured in a Hitachi F2500 spectrofluorimeter, and plots were fitted using the Grafit® software (Erithracus Software, Horley, Surrey, UK). All the assays were calculated by the Morrison equation for competitive inhibition (eq. 1, [Morrison, 1969]). The amount of substrate used for the tight binding titration experiments follow all the requirements, where [S]<<Kₘ for all enzymes, so the Kᵢₐₚₚ=Kᵢ.

$$V = SA \cdot ( {E}_{0} - \frac{( {E}_{0} + I + K i ) - \sqrt{{( {E}_{0} + I + K i )}^{2} - 4 \cdot {E}_{0} \cdot I}}{2} \qquad eq . 1$$

However, for furin we used a correction of K_{i app}, where [S]˜Kₘ (eq. 2).

Kᵢ=Kᵢₐₚₚ/1+[S]/Kₘ Eq. 2

Inhibition of Proteases with Alkylated Peptides (‘Geminoids’)

Range of Proteases Inhibited by Geminoids, Determination of Kᵢ with Z-RR-MCA (Dengue 2 Protease) and Ac-RVRR-MCA (Furin) (SEQ ID NO:60).

The first group of gemini-like peptide amphiphiles to be screened was that of C₁₆-K(G or A)ₙK-C₁₆ (compound type C, with R₁=n-C₁₅H₃₁, R₂=n-C₁₆H₃₃). Experiments on trypsin, thrombin, and plasmin are given in Table 4. The residual activity left upon inhibition with these geminoids decreased in the order trypsin≈thrombin>plasmin, but the inhibition of dengue 2 protease and human furin was even stronger; in addition to the results shown here, the geminoids also inhibited recombinant human thimet oligopeptidase (TOP), human cathepsin D, recombinant human cathepsin L, subtilisin A, and angiotensin converting enzyme with residual activities comparable to those of trypsin, and human kallikrein (substrate Abz-KLFSSKQ-EDDnp [Fogaça et al., 2001] SEQ ID NO:59) with Kᵢ values in the micromolar range as for dengue 2 protease and human furin. The results of the Kᵢ determination for trypsin, dengue 2 protease, and human furin are given in Table 1A; some general data for the inhibition by this type of compounds are given in Table 1B. For most compounds in Table 1 the Kᵢ could be derived from competitive inhibition experiments. Judging from the Kᵢ values with the relatively simple substrate Z-RR-MCA, the best inhibitors for dengue 2 protease contain alanine (A) rather than glycine (G).

TABLE 1A
Determination of Kᵢ for inhibition of the serine proteases trypsin, dengue
2 protease, and human furin (hFurin) by alkylated peptides
C₁₆-K(G or A)ₙK-C₁₆ (compound type C from FIG. 10, with
R₁ = n-C₁₅H₃₁, R₂ = n-C₁₆H₃₃)

| Inhibitor | C₁₆-K(G)ₙK-C₁₆ | | | C₁₆-K(A)ₙK-C₁₆ | | |
|---|---|---|---|---|---|---|
| n = | Trypsin | Dengue 2^{a)} | hFurin^{b)} | Trypsin | Dengue 2^{a)} | hFurin^{b)} |
| 0 | 84.70 | 5.37 | * | — | — | — |
| 1 | 41.60 | 1.56 | + | 15.20 | 0.37 | 0.54 |
| 2 | | 5.75 | + | | 0.63 | 0.97 |
| 3 | 11.10 | 2.50 | + | 40.30 | 1.41 | 1.72 |
| 4 | | 1.37 | + | | 2.40 | 1.75 |

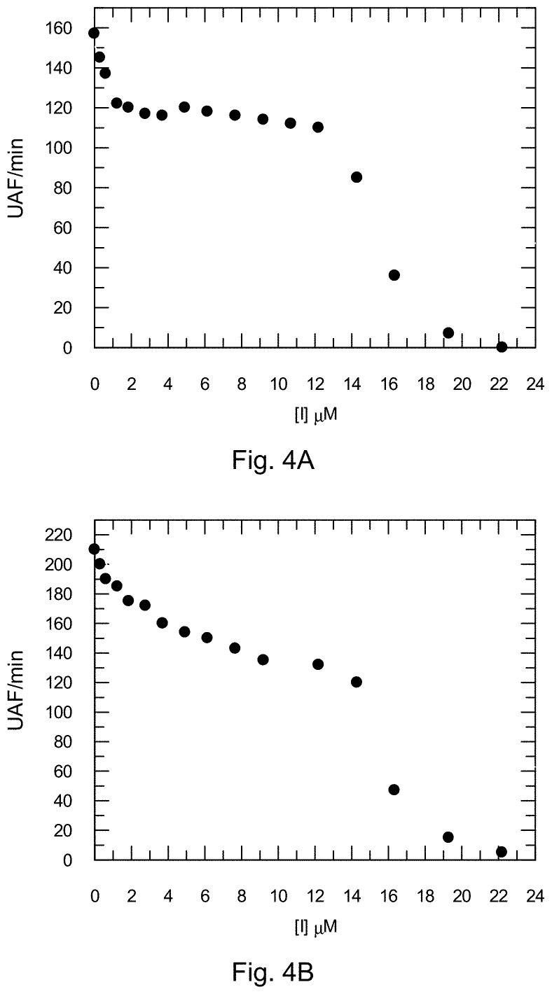

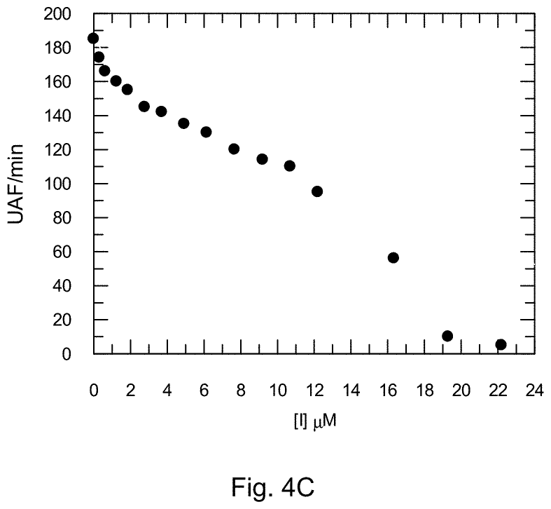

*no inhibition;
+ Kᵢ not determined; see the profiles in FIG. 4.
^{a)}In 50 mM Tris.HCl, pH 9.0, with 20 μM Z-RR-MCA, 37° C.
^{b)}In 10 mM Mes. NaOH, pH 7.0, with 2.35 μM Ac-RVRR-MCA (SEQ ID NO: 60), 37° C.

TABLE 1B
Inhibition of dengue 2 protease and furin by C₁₆-K(X)ₙ-K-C₁₆ with
X = G or A. These experiments were carried out in a Fluorescence
Spectrophotometer Hitachi F2500, 700 Volts, with λ_{exc} 380 nm and λₑₘ 460
nm for MCA substrates and λₑₓ 320 nm and λₑₘ 420 nm for Fluorescence
Resonance Energy Transfer (FRET) substrates, at 36.5° C.

| Inhibitors | Activity (%) | | Inhibitors | Activity (%) | |
|---|---|---|---|---|---|
| C16---- C16 | Dengue 2 | Furin | C16---- C16 | Dengue 2 | Furin |
| KK | | | | | |
| (20 μM) | 58 | 10 | | | |
| (50 μM) | 37 | 5 | | | |
| (100 μM) | 7 | 0 | | | |
| KGK | | | KAK | | |
| (5 μM) | 75 | 53 | (2.5 μM) | 87 | 43 |
| (10 μM) | 54 | 18 | (5 μM) | 66 | 30 |
| (15 μM) | 40 | 7 | (10 μM) | 26 | 10 |
| (20 μM) | 29 | 4 | (15 μM) | 15 | 0 |
| (40 μM) | 2 | 0 | (40 μM) | 6 | 0 |
| KGGK (SEQ ID NO: 41) | | | KAAK (SEQ ID NO: 42) | | |
| (5 μM) | 88 | 94 | (2.5 μM) | 89 | 94 |
| (10 μM) | 84 | 85 | (5 μM) | 79 | 83 |
| (15 μM) | 76 | 76 | (10 μM) | 68 | 77 |
| (20 μM) | 70 | 67 | (15 μM) | 45 | 69 |
| (40 μM) | 64 | 3 | (40 μM) | 10 | 3 |
| KGGGK (SEQ ID NO: 40) | | | KAAAK (SEQ ID NO: 52) | | |
| (5 μM) | 86 | 68 | (2.5 μM) | 91 | 73 |
| (10 μM) | 80 | 35 | (5 μM) | 85 | 56 |
| (15 μM) | 74 | 29 | (10 μM) | 75 | 24 |
| (20 μM) | 65 | 20 | (15 μM) | 64 | 10 |
| (40 μM) | 51 | 0 | (40 μM) | 25 | 0 |
| KGGGGK (SEQ ID NO: 61) | | | KAAAAK (SEQ ID NO: 53) | | |
| (5 μM) | 86 | 82 | (2.5 μM) | 94 | 70 |
| (10 μM) | 78 | 64 | (5 μM) | 90 | 59 |
| (15 μM) | 70 | 54 | (10 μM) | 85 | 52 |
| (20 μM) | 60 | 23 | (15 μM) | 79 | 45 |
| (40 μM) | 45 | 0 | (40 μM) | 45 | 0 |

Inhibition of Dengue 2 Protease Studied with Optimum Substrate.

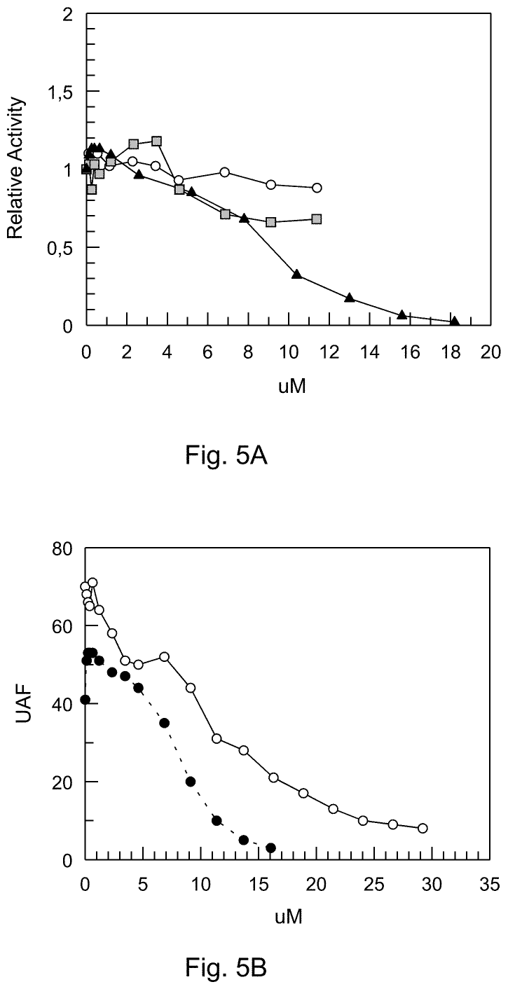

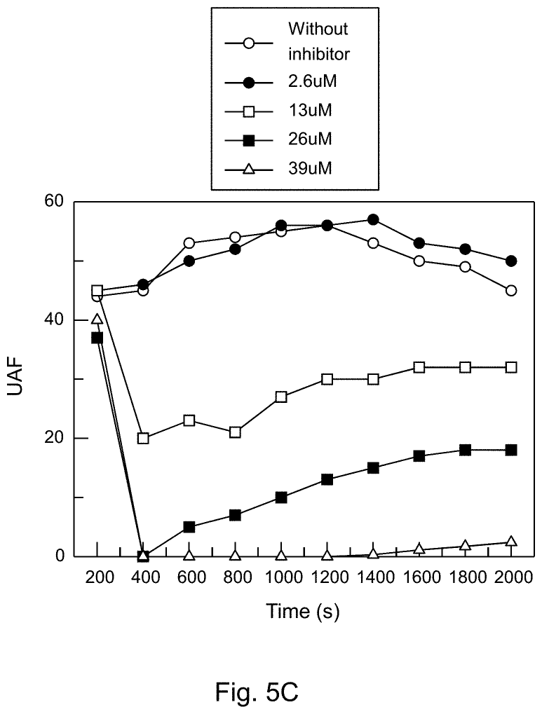

The best substrate for dengue protease is Abz-AKRR↓SQ-EDDnp (SEQ ID NO:1) [Gouvea et al., 2007]. The results of inhibition studies with this substrate are shown in FIG. 5. The data shown in FIG. 5a indicate that the geminoid with glycine (G) is a more effective Dengue protease inhibitor than the analogue with alanine (A); FIG. 5b shows that the inhibition by C₁₆-KGGK-C₁₆ (SEQ ID NO:41) does not depend on the choice of substrate. Geminoids with either G or A are inhibitors of Dengue protease, but which is the better inhibitor does depend on the nature of the substrates (compare FIG. 5a and Table 1).

Contrary to the experiments with furin and Ac-RVRR-MCA (SEQ ID NO:60) shown in FIG. 4, where the inhibitor gave a sharp decrease of enzyme activity at low concentration, low concentrations of inhibitor appear to have a stimulating effect on the conversion of Abz-AKRRSQ-EDDnp (SEQ ID NO:1) by Dengue protease in FIG. 5.

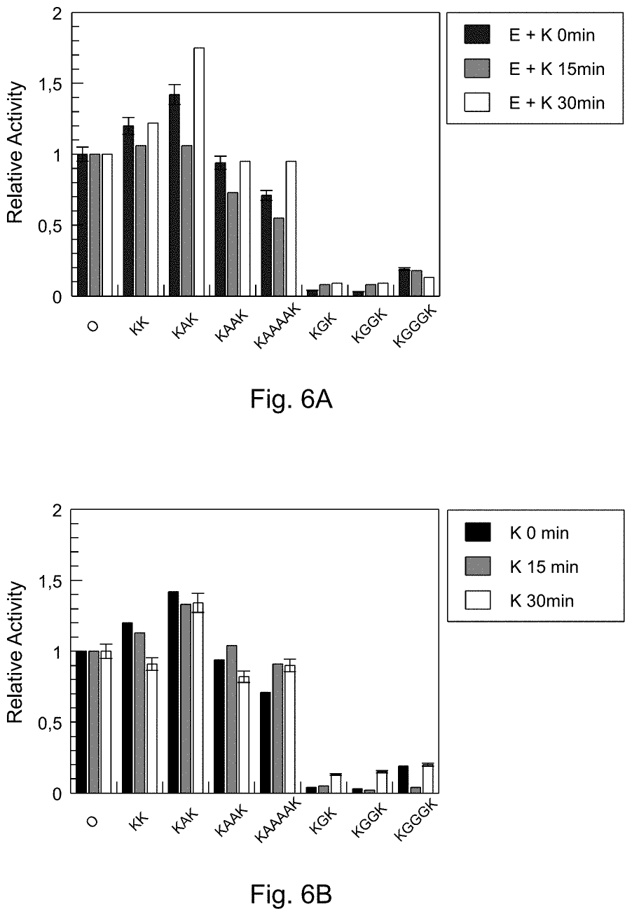

The effect of preincubation of the enzyme with inhibitor was studied in the experiments shown in FIG. 6. Preincubation of the geminoid inhibitors containing alanine led to a remarkable enhancement of the protease activity (FIG. 6a, left). Geminoids with glycine gave inhibition of enzymic activity which was gradually recovered after ½ hour (FIG. 6a, right). Enhancement after ½ hour of preincubation of the activity relative to zero time incubation makes sense in the context of the hypothesis that the inhibitor is partly hydrolysed and thereby rendered ineffective in the preincubation time. Preincubation of inhibitor alone had little effect on the inhibition by the alanine geminoids, but reduced the inhibition by the glycine geminoids (FIG. 6b).

TABLE 2
Inhibition of the hydrolysis of by dengue 2 protease of 10 μM
Z-RR-MCA in Tris.HCl pH 9.0, 20% glycerol, 37° C.

| | | | Activity | | | | |
|---|---|---|---|---|---|---|---|
| | | | with | | Activity | % | |
| | | Initial | inhibitors | | with | recovery in | |
| | Kᵢ^{a)} | activity ^{b)} | (μM of | | 0.05% | presence of | Nature |
| Inhibitor | (μM) | (UAF/min) | inhibitor) ^{b)} | | Triton ^{b)} | Triton ^{b)} | protocol ^{c)} |
| C₁₂KA₃KC₁₂ | 5.4 | 187 | 30 | (5 μM) | 85 | 45 | 0 |
| (SEQ ID NO: 52) | | | | | | | |
| C₁₂KA₂KC₁₂ | 11.6 | 228 | 5 | (20 μM) | 216 | 95 | 1 |
| (SEQ ID NO: 42) | | | | | | | |
| C₁₂KKC₁₂ | 132 | 200 | 0 | (30 μM) | 180 | 82 | 0 |
| C₁₆RARC₁₆ | 3.1 | 202 | 27 | (5 μM) | 105 | 52 | 0 |
| C₁₆RA₂RC₁₆ | 2.1 | 210 | 0 | (30 μM) | 128 | 61 | 0 |
| (SEQ ID NO: 56) | | | | | | | |
| C₁₆RA₃RC₁₆ | 1.3 | 250 | 55 | (15 μM) | 124 | 50 | 0 |
| (SEQ ID NO: 57) | | | | | | | |
| C₁₆RA₄RC₁₆ | 1.4 | 240 | 22 | (20 μM) | 164 | 68 | 2 |
| (SEQ ID NO: 58) | | | | | | | |
| C₁₆RRC₁₆ | 0.8 | 200 | 48 | (20 μM) | 148 | 74 | 0 |
| C₁₆KAKC₁₆ | 0.37 | 220 | 0 | (30 μM) | 91 | 41 | 0 |
| C₁₆KA₂KC₁₆ | 0.63 | 180 | 0 | (25 μM) | 100 | 56 | 0 |
| (SEQ ID NO: 42) | | | | | | | |
| C₁₆KA₃KC₁₆ | 1.41 | 182 | 0 | (30 μM) | 91 | 50 | 0 |
| (SEQ ID NO: 52) | | | | | | | |
| C₁₆KA₄KC₁₆ | 2.40 | 198 | 47 | (20 μM) | 133 | 67 | 0 |
| (SEQ ID NO: 53) | | | | | | | |
| C₁₆KKC₁₆ | 5.37 | 220 | 48 | (30 μM) | 113 | 52 | 1.5 |
| C₁₆KGKC₁₆ | 1.56 | 187 | 50 | (25 μM) | 90 | 48 | 0 |
| C₁₆KG₂KC₁₆ | 5.75 | 220 | 10 | (10 μM) | 160 | 73 | 0 |
| (SEQ ID NO: 41) | | | | | | | |
| C₁₆KG₃KC₁₆ | 2.50 | 206 | 40 | (30 μM) | 154 | 75 | 0 |
| (SEQ ID NO: 40) | | | | | | | |
| C₁₆KG₄KC₁₆ | 1.37 | 200 | 0 | (30 μM) | 125 | 63 | 0 |
| (SEQ ID NO: 61) | | | | | | | |

^{a)}Experimental procedure: 1) buffer + enzyme were pre-equilibrated for 2 min in cuvette at 37° C., 2) substrate was added and the hydrolysis measured for 200 s, 3) inhibitor was then added in different amounts.
^{b)} Procedure: the enzyme activity was determined in absence and in presence of inhibitor,
with and without 0.05% Triton X-100
^{c)} Procedure derived from the Nature Protocol [Feng & Shoichet, 2006]: 1) pre-incubation of buffer, enzyme, 0.01% Triton X-100, and inhibitor at 37° C., 2) substrate added after 5 min., 3) same procedure repeated without inhibitor.

2. Inhibition with Gemini Surfactants

In the gemini surfactants of type B (Kirby et al., 2003; FIG. 10) the peptide spacer was substituted by a methylene chain compared to the gemini-like surfactants according to formula I. This change led to some solubility problems, and stock solutions in 100% dimethylsulfoxide were prepared to solubilize the material. The Kᵢ parameters found for dengue 2 protease and human furin inhibition are shown in Table 3. It is found that this class of compounds are significantly poorer inhibitors of the proteases, as reflected in the higher Kᵢ compared to those found in the above for the geminoids. The compounds with R_{g}=C₁₁H₂₃ are the best inhibitors for dengue 2 protease, with the lowest Kᵢ for n=2 (12-2-12; R_{g}=C₁₁H₂₃, n=2) followed by n=4 (12-4-12). In the few cases where inhibition for human furin was also tested, a significantly different Kᵢ was measured, resulting in a stronger inhibition of furin for 12-6-12 (R_{g}=C₁₁H₂₃, n=6), and of dengue 2 for 12-4-12 (R_{g}=C₁₁H₂₃, n=4).

TABLE 3
Kᵢ parameters of inhibition of dengue 2 protease and human
furin (hFurin) by gemini surfactants of type B (FIG. 10).

| | 12-4-12 | 12-4-12 | 12-2-12 | 14-2-14 | 16-6-16 | 12-6-12 | 10-6-10 | 8-2-8 |
|---|---|---|---|---|---|---|---|---|
| R_{g} | C₁₁H₂₃ | C₁₁H₂₃ | C₁₁H₂₃ | C₁₃H₂₇ | C₁₅H₃₁ | C₁₁H₂₃ | C₉H₁₉ | C₇H₁₅ |
| N | 4 | 4 ^{a)} | 2 | 2 | 2 | 6 | 6 | 2 |
| Dengue 2 | 2.11 | 3.12 | 1.30 | 3.33 | 2.56 | 3.10 | 3.45 | 4.54 |
| hFurin | 5.90 | | | | | 1.60 | | |

^{a)} Extra Lys connected by amide bonds to the —NH₂ in the head groups.

Inhibition of Dengue Virus Replication in VERO Cells by C16-KAK-C16

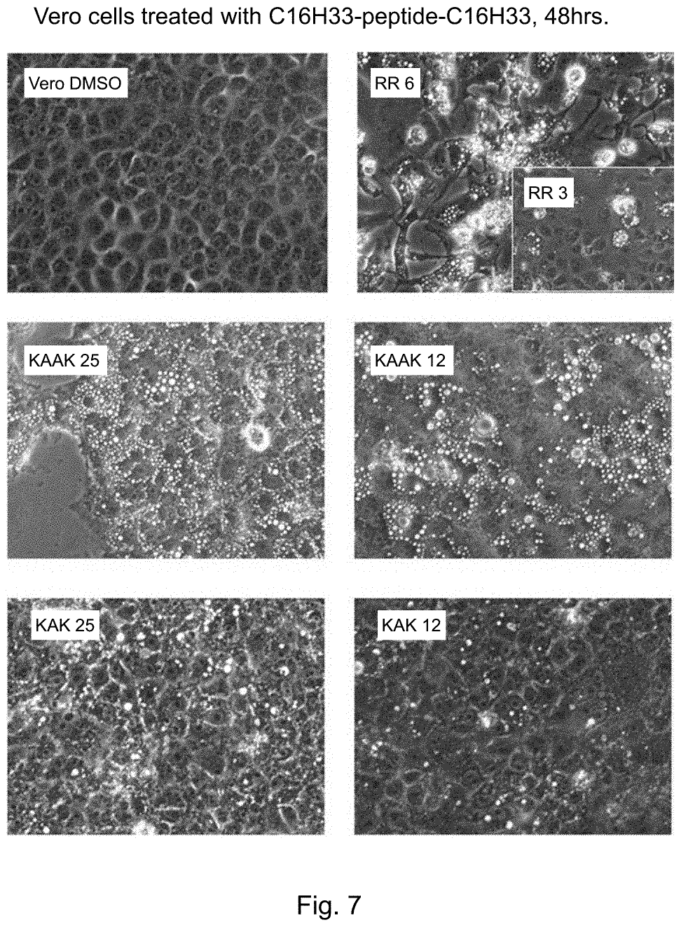

Based on the apparent Ki data obtained in vitro (Table 1A), we selected C16-K(A)ₙK-C16 for a test of dengue virus replication in cell in culture. DENV-2/NGC and VERO cells were used for the experiment. First, a toxicity study was performed in VERO cells with different Geminoids (FIG. 7). The results show that the compound with the highest relative affinity for Dengue virus protease (C16-KAK-C16) has the lowest effect on cell morphology in the effective concentration range, compared to C16-KAAK-C16 (SEQ ID NO:42) which induced substantial accumulation of intracellular vacuoles.

C16-KAK-C16 was subsequently tested in a standard Dengue replication (plaque) assay in VERO cells (FIG. 8). The data show that C16-KAK-C16 inhibits up to 90% of plaque formation depending on the viral load. In a separate experiment this was confirmed with a constant intermediate viral load of and different concentrations of C16-KAK-C16 (FIG. 9)

The results show an differential effect of different geminoids on cell phenotype (FIG. 7). Importantly, this suggests that the active geminoids, readily access intracellular membrane compartments, as predicted. Further, significant reduction of virus replication with little effect on cellular morphology was observed with the geminoid lead compound C16-KAK-C16, consistent with the in vitro inhibition data obtained separately (Table 1A).

### Discussion, Conclusions, and Outlook

The best inhibitor for dengue 2 protease of the lysine-based gemini surfactants (B) type (see FIG. 10) is 12-2-12 (Kᵢ 1.30 μM), followed by 12-4-12 (for explanation of this short notation see FIG. 10) with Kᵢ values in the low micromolar range. The geminoids (gemini-like peptide amphiphiles) of type C₁₆-KAₙK-C₁₆ are even stronger inhibitors, with Kᵢ values for dengue 2 protease of below the micromolar range. A strong indication that the aggregation behaviour of this type of compounds is important for the inhibition came from the attempts to determine the Kᵢ for the inhibition of furin by C₁₆-KGₙK-C₁₆; the dependence between residual activity on inhibitor concentration was not linear, but instead showed a disproportional decrease above a concentration of approx. 12 μM, which presumably corresponds to the critical micelle concentration. The assessment which geminoid is the stronger or more selective inhibitor is complicated by this aggregation, which appears to be stronger for geminoids with Gly than for Ala; with Z-RR-MCA as the substrate, the Ala geminoids are better inhibitors for dengue protease as judged by Kᵢ, but with Abz-AKRRSQ-EDDnp (SEQ ID NO:1) the Gly geminoids are stronger as judged from the activity/inhibitor concentration profile. In the presence of 0.01% of the non-ionic surfactant Triton X-100, which disrupts the aggregates of other surfactants, the inhibition of dengue 2 protease was virtually completely abolished, with small (1-2%) residual inhibition left for C₁₂-KA₂K-C₁₂ (SEQ ID NO:42), C₁₆-KA₄K-C₁₆ (SEQ ID NO:53), and C₁₆-KK-C₁₆. Although the protocol with Triton X-100 [Feng & Shoichet, 2006] was designed to rule out ‘promiscuous’ or ‘non-specific’ inhibition, it can not be concluded that the inhibitors are non-specific. First, they do display a dependence of the inhibition on the structure peptide part when their aggregates are not disrupted. Further, considering the amphiphilic nature of the geminoids it is perfectly conceivable that they dissolve in the Triton micro-micelles in such a way that the peptide element can no longer interact with the active center of the protease. A further investigation of the interactions of the geminoid inhibitors with biological membranes and with membrane-bound proteases is needed. The inhibition of the proteases by peptide amphiphile aggregates could be a matter of proper presentation of the inhibitory peptide sequence on the hydrophilic surface of the aggregate to the enzyme (implying specifity for the peptide sequence, as observed), instead of inhibition by an a specific sequestration of the enzyme inside the aggregate. An indication for a specific interaction is the finding that the enzyme activity is regained upon prolonged incubation with the inhibitor; the explanation for this result is that the inhibitors are actually (poor) substrates for the enzyme and are degraded to products that are poorer inhibitors, either because they no longer contain sufficient cationic (K, R) residues to be recognized by the enzyme, or because they have lost one of their alkyl tails so that they have become classical rather than geminoid surfactants, with correspondingly higher critical aggregation concentrations. In view of the apparent effect of the aggregation on the efficiency of the inhibition it would be of interest to investigate the aggregation behavior of the inhibitors in the presence and absence of the protein, but also to prepare compounds of the types i) -KAK- peptide with only the carboxyl terminal alkylated, and ii) analogous compounds with shorter alkyl tails (5-10 C atoms). The preparation and tests of peptides symmetrically, asymmetrically and singly alkylated with saturated, short (C6), and unsaturated (C18:1) alkyl tails are in progress.

The amphiphilic nature of the inhibitor could have various advantages for their application as drugs, such as formation of nanoparticles in the blood, and the possible translocation into the cell. Cationic peptides are also studied for their antimicrobial properties, and their ability to penetrate the cell as nanoparticles with the cationic membrane translocation ‘TAT’ peptide sequence on the outside [Liu et al., 2009] is an important factor in their efficiency. It is not unlikely that the amphiphilic cationic peptides can be taken up by the cell by endocytosis, analogous to what has been proposed for lipoplexes with cationic gemini surfactants in transfection [Kirby et al., 2003; Bell et al., 2003]. Indeed, the effect of the geminoids on cell morphology at high concentration (FIG. 7), is consistent with an effect on intracellular processing enzymes, in particular Furring (Table 1A) Compared to the situation where the cationic surfactants are bound to a polynucleotide, there is a larger chance that they will be integrated into the biological membrane. The question whether the amphiphilic peptides can be delivered to the cell and/or the virus and if so, what would be the effect of their aggregation state and the effect of fusogenic lipids (e.g. DOPE, dioleoylphosphatidyl ethanolamine) is an important and challenging subject of study. Dengue virus titration assays of the most important compound reported here confirmed that significant inhibition of Dengue replication can be achieved in cells in culture (FIG. 8,9). Further studies to optimise the peptide part and the alkyl tails, to achieve higher activity, selectivity and delivery to the relevant cellular compartment are in progress. Unsaturated alkyl tails, have been established to be more efficient for delivery of nucleic acids to cytoplasm than unsaturated alkyl tails [Kirby et al., 2003; Bell et al., 2003; Damen et al., 2010].

TABLE 4
Inhibition of serine proteases by C₁₆-K(X)ₙ-K-C₁₆
with X = G or A. These experiments were carried
out in a Fluorescence Spectrophotometer
Hitachi F2500, 700 Volts, with λ_{exc} 380 nm and
λₑₘ 460 nm for MCA substrates and λₑₓ 320 nm and
λₑₘ 420 nm for Fluorescence Resonance
Energy Transfer (FRET) substrates, at 36.5° C.

| Inhibitors | Residual Activity (%) | | |
|---|---|---|---|
| C16----- C16 | Trypsin | Thrombin | Plasmin |
| KK | | | |
| (15 μM) | 82 | 62 | 39 |
| (55 μM) | 58 | 48 | 11 |
| (85 μM) | 43 | 37 | 4 |
| KGK | | | |
| (15 μM) | 76 | 75 | |
| (55 μM) | 40 | 61 | |
| (85 μM) | 24 | 40 | |
| KGGK (SEQ ID NO: 41) | | | |
| (18 μM) | 79 | 78 | 30 |
| (55 μM) | 45 | 46 | 11 |
| (85 μM) | 24 | 33 | 4 |
| KGGGK (SEQ ID NO: 40) | | | |
| (18 μM) | 84 | 81 | |
| (55 μM) | 56 | 64 | |
| (85 μM) | 38 | 45 | |
| KGGGGK (SEQ ID NO: 61) | | | |
| (18 μM) | 81 | 88 | |
| (55 μM) | 67 | 59 | |
| (85 μM) | 53 | 41 | |
| KAK | | | |
| (15 μM) | 68 | 80 | 62 |
| (40 μM) | 35 | 64 | 20 |
| (80 μM) | 21 | 42 | 3 |
| KAAK (SEQ ID NO: 42) | | | |
| (15 μM) | 64 | 80 | |
| (40 μM) | 20 | 55 | |
| (80 μM) | 7 | 39 | |
| KAAAK (SEQ ID NO: 52) | | | |
| (15 μM) | 79 | | |
| (40 μM) | 54 | | |
| (80 μM) | 40 | | |
| KAAAAK (SEQ ID NO: 53) | | | |
| (15 μM) | 73 | | |
| (40 μM) | 35 | | |
| (80 μM) | 20 | | |

### Example 3: Activity of Geminoid Inhibitors in DENV2 Protease Assays

Table activity of inhibitors in different
DENV2 protease replicon assay (1) and DENV2
protease biochemical assays (2)

| | Replicon | | IC₅₀ |
|---|---|---|---|
| | activity | % Inhibition | [μM] |
| Inhibitor | [μM] (1) | at 50 μM (2) | (2) |
| C16-AAKK-C16 | 5.0 | | |
| (SEQ ID NO: 62) | | | |
| C16-LAKK-C16 | 5.0 | | |
| (SEQ ID NO: 63) | | | |
| C16-FAKK-C16 | 3.0 | | |
| (SEQ ID NO: 64) | | | |
| C16-ALKK-C16 | 10.0 | | |
| (SEQ ID NO: 65) | | | |
| C16-LLKK-C16 | 3.0 | | |
| (SEQ ID NO: 66) | | | |
| C16-FLKK-C16 | 3.0 | | |
| (SEQ ID NO: 67) | | | |
| C16-AFKK-C16 | 5.0 | | |
| (SEQ ID NO: 68) | | | |
| C16-LFKK-C16 | 10.0 | | |
| (SEQ ID NO: 69) | | | |
| C16-FFKK-C16 | 5.0 | | |
| (SEQ ID NO: 70) | | | |
| C16-AKKK-C16 | 1.0 | | |
| (SEQ ID NO: 71) | | | |
| C8-AKKK-C8 | 10.0 | | |
| (SEQ ID NO: 71) | | | |
| C8-LKKK-C8 | 10.0 | | |
| (SEQ ID NO: 72) | | | |
| C8-FKKK-C8 | 10.0 | | |
| (SEQ ID NO: 73) | | | |
| C3-AKKK-C3 | 10.0 | | |
| (SEQ ID NO: 71) | | | |
| C3-LKKK-C3 | 10.0 | | |
| (SEQ ID NO: 72) | | | |
| C3-FKKK-C3 | 10.0 | | |
| (SEQ ID NO: 73) | | | |
| C16-LKKK-C16 | 1.0 | | |
| (SEQ ID NO: 72) | | | |
| C16-FKKK-C16 | 5.0 | 95.8 | 0.2 |
| (SEQ ID NO: 73) | | | |
| C20-AKKK-C20 | 3.0 | | |
| (SEQ ID NO: 71) | | | |
| C20-LKKK-C20 | 1.0 | | |
| (SEQ ID NO: 72) | | | |
| C20-FKKK-C20 | 0.9 | | |
| (SEQ ID NO: 73) | | | |
| C12-AKKK-C12 | 0.1 | 98.5 | 0.3 |
| (SEQ ID NO: 71) | | | |
| C12-LKKK-C12 | 0.1 | 101.6 | 0.3 |
| (SEQ ID NO: 72) | | | |
| C12-FKKK-C12 | 10.0 | | |
| (SEQ ID NO: 73) | | | |
| C16-AKAK-C16 | 3.0 | 103.7 | |
| (SEQ ID NO: 74) | | | |
| C16-LKAK-C16 | 3.0 | | |
| (SEQ ID NO: 75) | | | |
| C12-AKAK-C12 | 10.0 | | |
| (SEQ ID NO: 74) | | | |
| C12-LKAK-C12 | 10.0 | | |
| (SEQ ID NO: 75) | | | |
| C8-AKAK-C8 | 10.0 | | |
| (SEQ ID NO: 74) | | | |
| C8-LKAK-C8 | 10.0 | | |
| (SEQ ID NO: 75) | | | |
| C2-AKAK-C3 | 10.0 | | |
| (SEQ ID NO: 74) | | | |
| C2-LKAK-C3 | 10.0 | | |
| (SEQ ID NO: 75) | | | |
| C16-ARRK-C16 | 0.3 | | |
| (SEQ ID NO: 76) | | | |
| C16-RARK-C16 | 3.0 | | |
| (SEQ ID NO: 77) | | | |
| C12-ARRK-C12 | 0.3 | | |
| (SEQ ID NO: 76) | | | |
| C12-RARK-C12 | 1.0 | | |
| (SEQ ID NO: 77) | | | |
| C8-ARRK-C8 | 10.0 | | |
| (SEQ ID NO: 76) | | | |
| C3-ARRK-C3 | 3.0 | | |
| (SEQ ID NO: 76) | | | |
| C16-AAQK-C16 | 5.0 | 26.8 | |
| (SEQ ID NO: 78) | | | |
| C16-RAQK-C16 | 10.0 | 101.7 | 0.6 |
| (SEQ ID NO: 79) | | | |
| C16-ARQK-C16 | 5.0 | 95.7 | 0.3 |
| (SEQ ID NO: 80) | | | |
| C16-LLQK-C16 | 0.3 | | |
| (SEQ ID NO: 81) | | | |
| C16-FFQK-C16 | 3.0 | | |
| (SEQ ID NO: 82) | | | |
| C16-LKRR-C16 | 3.0 | | |
| (SEQ ID NO: 83) | | | |
| C16-GLKR-C16 | 3.0 | | |
| (SEQ ID NO: 84) | | | |
| C16-QRKR-C16 | 3.0 | | |
| (SEQ ID NO: 85) | | | |
| C16-NKKR-C16 | 3.0 | | |
| (SEQ ID NO: 86) | | | |
| C16-KAK-C16 | 0.3 | 105.6 | |
| C16-KAAK-C16 | 3.0 | 97.7 | |
| (SEQ ID NO: 42) | | | |
| C16-KGK-C16 | 1.0 | 102.9 | |
| C16-KGGK-C16 | 0.3 | 103.9 | |
| (SEQ ID NO: 41) | | | |
| C16-D-AKAK-C16 | 2.9 | | |
| (SEQ ID NO: 74) | | | |
| C16-D-LKAK-C16 | 9.9 | | |
| (SEQ ID NO: 75) | | | |
| C16-D-AKAKG- | 3.0 | | |
| C16 | | | |
| (SEQ ID NO: 87) | | | |
| C16-D-LKAKG- | 3.0 | | |
| C16 | | | |
| (SEQ ID NO: 88) | | | |
| C16-D-AKAKS-C16 | 5.0 | | |
| (SEQ ID NO: 89) | | | |
| C16-D-LKAKS-C16 | 3.0 | 97.4 | 0.5 |
| (SEQ ID NO: 90) | | | |
| C16-D-AKAK-L-S- | 3.0 | 108.0 | |
| C16 | | | |
| (SEQ ID NO: 89) | | | |
| C16-D-LKAK-L-S- | 3.0 | | |
| C16 | | | |
| (SEQ ID NO: 90) | | | |
| C16-AKAKS-C16 | 1.0 | | |
| (SEQ ID NO: 89) | | | |
| C16-LKAKS-C16 | 3.0 | 108.0 | 0.3 |
| (SEQ ID NO: 90) | | | |
| C16-AKAKG-C16 | 3.0 | 102.3 | |
| (SEQ ID NO: 87) | | | |
| C16-LKAKG-C16 | 3.0 | 98.4 | |
| (SEQ ID NO: 88) | | | |

(1) lowest concentration with observed activity in replicon assay, according to: Masse et al., Antiviral Research 86, 3 (2010) 296-305; Kato and Hishiki, Viruses (2016) 8, 122
(2) acccording to: Steuer et al., Journal of Biomolecular Screening 14, 9 (2009) 1102-1108; Sebaugh, Pharmaceutical statistics (2010)

### REFERENCES

W. F. Anderson, “Prospects for Human Gene Therapy.” Science 226 (1984) 401-409.
P. C. Bell, M. Bergsma, I. P. Dolbnya, W. Bras, M. C. A. Stuart, A. E. Rowan, M. C. Feiters, J. B. F. N. Engberts, Transfection Mediated by Gemini Surfactants: Engineered Escape from the Endosomal Compartment. J. Am. Chem. Soc. 125 (2003) 1551-1558.
K. Berns, E. M. Hijmans, J. Mullenders, T. R. Brummelkamp, A. Velds, M. Heimerikx, R. M. Kerkhoven, M. Madiredjo, W. Nijkamp, B. Weigelt, R. Agami, W. Ge, G. Cavet, P. S. Linsley, R. L. Beijersbergen, R. Bernards, “A large-scale RNAi screen in human cells identifies new components of the p53 pathway.” Nature 428 (2004) 431-437.
M. Damen, PhD Thesis in preparation, Radboud University Nijmegen
M. Damen, J. Aarbiou, S. F. M. van Dongen, R. M. Buijs-Offerman, P. P. Spijkers, M. van den Heuvel, K. Kvashnina, R. J. M. Nolte, B. J. Scholte, M. C. Feiters, “Delivery of DNA and siRNA by novel gemini-like amphiphilic peptides.” J. Controlled Release 145 (2010) 33-39.
M. Damen, S. F. M. van Dongen, J. Aarbiou, P. P. Spijkers, B. J. Scholte, M. C. Feiters, R. J. M. Nolte, “Gemini-Like Peptide-Based Amphiphiles for Application in Gene Transfection”. Human Gene Therapy (2007) 18, 1052 (Proceedings of the XVth Annual Congress of the European Society of Gene and Cell Therapy, Oct. 27-30, 2007, Rotterdam, The Netherlands, P 154).
P. Erbel, N. Schiering, A. D'Arcy, M. Renatus, M. Kroemer, S. P. Lim, Z. Yin, T. H. Keller, S. G. Vasudevan, U. Hommel, Nat. Struct. Mol. Biol. 13 (2006) 372-373. “Structural basis for the activation of flaviviral NS3 proteases from dengue and West Nile virus”.
B. Y. Feng, A. Shelat, T. N. Doman, R. K. Guy, B. K. Shoichet, “High-throughput assays for promiscuous inhibitors.” Nature Chem. Biol. 1 (2005) 146-148.
B. Y. Feng, B. K. Shoichet, “A detergent-based assay for the detection of promiscuous inhibitors”, Nature Protocols 1 (2006) 550-553.
S. E. Fogaça, R. L. Melo, D. C. Pimenta, K. Hosoi, L. Juliano, M. A. Juliano, “Differences in substrate and inhibitor sequence specificity of human, mouse and rat tissue kallikreins”, Biochem. J. 380 (2004) 775-781.
I. E. Gouvea, M. A. Izidoro, W. A. S. Judice, M. H. S. Cezari, G. Caliendo, V. Santagada, C. N. D. dos Santos, M. H. Queiroz, M. A. Juliano, P. R. Young, D. P. Fairlie, L. Juliano, “Substrate specificity of recombinant dengue 2 virus NS2B-NS3 protease: Influence of natural and unnatural basic amino acids on hydrolysis of synthetic fluorescent substrates.” Arch. Biochem. Biophys. 457 (2007) 187-196.
S. Henrich, A. Cameron, G. P. Bourenkov, R. Kiefersauer, R. Huber, I. Lindberg, W. Bode, M. E. Than, “The crystal structure of the proprotein processing proteinase furin explains its stringent specificity”. Nature Struct. Biol. 10 (2003) 520-526.
M. A. Izidoro, I. E. Gouvea, J. A. N. Santos, D. M. Assis, V. Oliveira, W. A. S. Judice, M. A. Juliano, I. Lindberg, L. Juliano. “Human furin hydrolytic specificity on synthetic peptides derived from processing sites in virus polyproteins and cellular proproteins: —effects of potassium and magnesium ions”. Arch. Biochem. Biophys. 487 (2009) 105-114.
A. J. Kirby, P. Camilleri, J. B. F. N. Engberts, M. C. Feiters, R. J. M. Nolte, O. Söderman, M. Bergsma, P. C. Bell, M. L. Fielden, C. L. García Rodriguez, P. Guédat, A. Kremer, C. McGregor, C. Perrin, G. Ronsin, M. C. P. van Eijk, “Gemini Surfactants: New Synthetic Vectors for Gene Transfection.”.Angew. Chem. Int. Ed. Engl. 42 (2003) 1448-1457.
D. Leung, K. Schroder, H. White, N.-X. Fang, M. J. Stoermer, G. Abbenante, J. L. Martin, P. R. Young, D. P. Fairlie, “Activity of Recombinant Dengue 2 Virus NS3 Protease in the Presence of a Truncated NS2B Co-factor, Small Peptide Substrates, and Inhibitors.” J. Biol. Chem. 276 (2001) 45762-45771.
L. Liu, K. Xu, H. Wang, P. K. J. Tan, W. Fan, S. S. Venkatraman, L. Li, Y.-Y. Yang, “Self-assembled cationic peptide nanoparticles as an efficient antimicrobial agent.” Nature Nanotechnology 4 (2009) 457-463.
D. Luo, T. Xu, C. Hunke, G. Grüber, S. G. Vasudevan, J. Lescar, “Crystal Structure of the NS3 Protease-Helicase from Dengue Virus”. J. Virol. 82 (2008) 173-183.
R. J. Melo, R. C. Barbosa Pozzo, L. C. Alves, E. Perissutti, G. Caliendo, V. Santagada, L. Juliano, M. A. Juliano, “Synthesis and hydrolysis by cathepsin B of £uorogenic substrates with the general structure benzoyl-X-ARG-MCA containing non-natural basic amino acids at position X”. Biochim. Biophys. Acta 1547 (2001) 82-94.
F. M. Menger & J. S. Keiper, “Gemini surfactants.” Angew Chem Int Ed Engl 39 (2000) 1906
J. F. Morrison, “Kinetics of the reversible inhibition of enzyme-catalysed reactions by tight-binding inhibitors.” Biochim Biophys Acta. 185 (1969) 269-286.
H. Ten Brink, J. T. Meijer, R. V. Geel, M. Damen, D. W. P. M. Löwik, J. C. M. van Hest, “Solid-phase synthesis of C-terminally modified peptides.” J. Pept. Sci. 12 (2006) 686-692 (2006).
J. L. Wheatley, T. Holyoak, “Differential P1 arginine and lysine recognition in the prototypical proprotein convertase Kex2”. Proc. Natl. Acad. Sci. USA 104 (2007) 6626-6631.

### Further Literature Cited

[1] Pruessmeyer J, Ludwig A. The good, the bad and the ugly substrates for ADAM10 and ADAM17 in brain pathology, inflammation and cancer. Semin Cell Dev Biol. 2009 April; 20(2):164-74.
[2] Arribas J, Esselens C. ADAM17 as a therapeutic target in multiple diseases. Curr Pharm Des. 2009; 15(20):2319-35.
[3] Kenny P A. Tackling EGFR signaling with TACE antagonists: a rational target for metalloprotease inhibitors in cancer. Expert Opin Ther Targets. 2007 October; 11(10):1287-98.
[4] Caescu C I, Jeschke G R, Turk B E. Active site determinants of substrate recognition by the metalloproteinases TACE and ADAM10. Biochem J. 2009 Aug. 28.
[5] DasGupta S, Murumkar P R, Giridhar R, Yadav M R. Current perspective of TACE inhibitors: a review. Bioorg Med Chem. 2009 Jan. 15; 17(2):444-59.
[6] Tiddens H A. Detecting early structural lung damage in cystic fibrosis. Pediatric pulmonology. 2002 September; 34(3):228-31.
[7] de Jong P A, Lindblad A, Rubin L, Hop W C, de Jongste J C, Brink M, Tiddens H A. Progression of lung disease on computed tomography and pulmonary function tests in children and adults with cystic fibrosis. Thorax. 2006 January; 61(1):80-5.
[8] Tiddens H A, Donaldson S H, Rosenfeld M, Pare P D. Cystic fibrosis lung disease starts in the small airways: can we treat it more effectively? Pediatric pulmonology. 2010 February; 45(2):107-17.
[9] Meyer M, Huaux F, Gavilanes X, van den Brule S, Lebecque P, Lo Re S, Lison D, Scholte B, Wallemacq P, Leal T. Azithromycin reduces exaggerated cytokine production by ml alveolar macrophages in cystic fibrosis. American journal of respiratory cell and molecular biology. 2009 November; 41(5):590-602.
[10] Bruscia E M, Zhang P X, Ferreira E, Caputo C, Emerson J W, Tuck D, Krause D S, Egan M E. Macrophages directly contribute to the exaggerated inflammatory response in cystic fibrosis transmembrane conductance regulator−/− mice. American journal of respiratory cell and molecular biology. 2009 March; 40(3):295-304.
[11] Di A, Brown M E, Deriy L V, Li C, Szeto F L, Chen Y, Huang P, Tong J, Naren A P, Bindokas V, Palfrey H C, Nelson D J. CFTR regulates phagosome acidification in macrophages and alters bactericidal activity. Nat Cell Biol. 2006 September; 8(9):933-44.
[12] Adib-Conquy M, Pedron T, Petit-Bertron A F, Tabary O, Corvol H, Jacquot J, Clement A, Cavaillon J M. Neutrophils in Cystic Fibrosis Display a Distinct Gene Expression Pattern. Mol Med. 2007 Jun. 11.
[13] Cutting G R. Modifier genetics: cystic fibrosis. Annu Rev Genomics Hum Genet. 2005; 6:237-60.
[14] Banner K H, De Jonge H, Elborn S, Growcott E, Gulbins E, Konstan M, Moss R, Poll C, Randell S H, Rossi A G, Thomas L, Waltz D. Highlights of a workshop to discuss targeting inflammation in cystic fibrosis. J Cyst Fibros. 2009 January; 8(1):1-8.
[15] Gao H, Ward P A. STAT3 and suppressor of cytokine signaling 3: potential targets in lung inflammatory responses. Expert Opin Ther Targets. 2007 July; 11(7):869-80.
[16] Torosyan Y, al. E. Systems biology approach to gene expression profiling in bronchial epithelial cells from cystic fibrosis patients: influence of disease severety and gender. Pediatric Pulmomology. 2009; suppl 32:173 (abstract).
[17] Wilk J B, Walter R E, Laramie J M, Gottlieb D J, O'Connor G T. Framingham Heart Study genome-wide association: results for pulmonary function measures. BMC medical genetics. 2007; 8 Suppl 1:S8.
[18] Edmiston J S, Archer K J, Scian M J, Joyce A R, Zedler B K, Murrelle E L. Gene expression profiling of peripheral blood leukocytes identifies potential novel biomarkers of chronic obstructive pulmonary disease in current and former smokers. Biomarkers. December; 15(8):715-30.
[19] Horiuchi K, Kimura T, Miyamoto T, Takaishi H, Okada Y, Toyama Y, Blobel C P. Cutting edge: TNF-alpha-converting enzyme (TACE/ADAM17) inactivation in mouse myeloid cells prevents lethality from endotoxin shock. J Immunol. 2007 Sep. 1; 179(5):2686-9.
[20] Legssyer R, Huaux F, Lebacq J, Delos M, Marbaix E, Lebecque P, Lison D, Scholte B J, Wallemacq P, Leal T. Azithromycin reduces spontaneous and induced inflammation in DeltaF508 cystic fibrosis mice. Respiratory research. 2006; 7:134.
[21] Burgel P R, Nadel J A. Roles of epidermal growth factor receptor activation in epithelial cell repair and mucin production in airway epithelium. Thorax. 2004 November; 59(11):992-6.
[22] Perugorria M J, Latasa M U, Nicou A, Cartagena-Lirola H, Castillo J, Goni S, Vespasiani-Gentilucci U, Zagami M G, Lotersztajn S, Prieto J, Berasain C, Avila M A. The epidermal growth factor receptor ligand amphiregulin participates in the development of mouse liver fibrosis. Hepatology. 2008 October; 48(4):1251-61.
[23] Korfhagen T R, Le Cras T D, Davidson C R, Schmidt S M, Ikegami M, Whitsett J A, Hardie W D. Rapamycin prevents transforming growth factor-alpha-induced pulmonary fibrosis. American journal of respiratory cell and molecular biology. 2009 November; 41(5):562-72.
[24] Gomez M I, Sokol S H, Muir A B, Soong G, Bastien J, Prince A S. Bacterial induction of TNF-alpha converting enzyme expression and IL-6 receptor alpha shedding regulates airway inflammatory signaling. J Immunol. 2005 Aug. 1; 175(3):1930-6.
[25] Chalaris A, Rabe B, Paliga K, Lange H, Laskay T, Fielding C A, Jones S A, Rose-John S, Scheller J. Apoptosis is a natural stimulus of IL6R shedding and contributes to the proinflammatory trans-signaling function of neutrophils. Blood. 2007 Sep. 15; 110(6):1748-55.
[26] Ammit A J, Moir L M, Oliver B G, Hughes J M, Alkhouri H, Ge Q, Burgess J K, Black J L, Roth M. Effect of IL-6 trans-signaling on the pro-remodeling phenotype of airway smooth muscle. American journal of physiology. 2007 January; 292(1):L199-206.
[27] Saito F, Tasaka S, Inoue K, Miyamoto K, Nakano Y, Ogawa Y, Yamada W, Shiraishi Y, Hasegawa N, Fujishima S, Takano H, Ishizaka A. Role of interleukin-6 in bleomycin-induced lung inflammatory changes in mice. American journal of respiratory cell and molecular biology. 2008 May; 38(5):566-71.
[28] Holgate S T. ADAM metallopeptidase domain 33 (ADAM33): identification and role in airways disease. Drug news & perspectives. July-August; 23(6):381-7.
[29] Pabst S, Pizarro Touron C, Gillissen A, Lennarz M, Tuleta I, Nickenig G, Skowasch D, Grohe C. ADAM33 gene polymorphisms in chronic obstructive pulmonary disease. European journal of medical research. 2009 Dec. 7; 14 Suppl 4:182-6.
[30] Cosgrove S, Chotirmall S H, Greene C M, McElvaney N G. Pulmonary proteases in the cystic fibrosis lung induce interleukin 8 expression from bronchial epithelial cells via a heme/meprin/epidermal growth factor receptor/Toll-like receptor pathway. The Journal of biological chemistry. March 4; 286(9):7692-704.
[31] Garcia-Caballero A, Ishmael S S, Dang Y, Gillie D, Bond J S, Milgram S L, Stutts M J. Activation of the epithelial sodium channel by the metalloprotease meprin beta subunit. Channels (Austin, Tex. January-February; 5(1):14-22.

1-18. (canceled)

19. A method of treatment or prophylaxis of a disease selected from the group consisting of viral infection, inflammation, and ADAM17-mediated diseases, the method comprising administering, to a subject in need thereof, a therapeutically or prophylactically effective amount of a compound according to Formula (I):
R¹—C(═O)—Zₙ—NR³-R² (I);
or a salt or solvate thereof,
in which R¹ and R² are each independently saturated, partly saturated or unsaturated, straight, branched or cyclic alkyl chains, wherein R¹ has a number of C atoms of 11 to 19, and R² has a number of C atoms of 12 to 20; R³ is hydrogen or C₁-C₆ alkyl;
n is an integer from 1 to 15;
each Z independently is an amino acid residue, wherein Zₙ comprises an N-terminus attached to C(═O) and a C-terminus that is attached to NR³.

20. The method according to claim 19, wherein R¹—C(═O) and R² each independently have a number of carbon atoms of 14 to 20.

21. The method according to claim 20, wherein R¹—C(═O) and R² each independently have a number of carbon atoms of 16 to 18.

22. The method according to claim 19, wherein the amino acid residue Z is a residue of an amino acid chosen from the group of natural amino acids, beta-alanine (bAla), 4-aminomethyl phenylalanine (Amf), 4-guanidine phenylalanine (Gnf), 4-aminomethyl-N-isopropyl phenylalanine (Iaf), 3-pyridyl alanine (Pya), 4-piperidyl alanine (Ppa), 4-aminomethyl cyclohexyl alanine (Ama), 4-aminocyclohexyl alanine (Aca), ornithine (Orn), citrulline, hydroxylysine (Hyl), allo-hydroxylysine (aHyl), 6-N-methyllysine (MeLys), desmosine (Des), isodesmosine (Ide), 2-aminoadipic acid (Aad), 3-aminoadipic acid (bAad), 2-aminobutyric acid (Abu), 4-aminobutyric acid (4Abu), 6-aminohexanoic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), 2,2′-diaminopimelic acid (Dpm), 2-3-diaminopropionic acid (Dpr), N-ethylglycine (EtGly), N-ethylasparagine (EtAsn), 3-hydroxyproline (3Hyp), 4-hydroxyproline (4Hyp), allo-isoleucine (AIle), sarcosine (MeGly), N-methylisoleucine (MeIle), N-methylvaline (MeVal), norvaline (Nva), and norleucine (Nle).

23. The method according to claim 22, wherein each amino acid residue Z independently is a residue of a natural amino acid.

24. The method according to claim 19, wherein n is an integer from 1 to 10.

25. The method according to claim 24, wherein n is an integer from 3 to 8.

26. The method according to claim 19, wherein R³ is H.

27. The method according to claim 19, wherein the alkyl chains are unsaturated.

28. The method according to claim 19, wherein R¹ comprises 11 to 13 carbon atoms, R² comprises 12 to 14 carbon atoms, and n is 4.

29. The method according to claim 19, wherein R¹ comprises 13 carbon atoms and R² comprises 14 carbon atoms.

30. The method according to claim 19, wherein Zₙ in the compound of Formula (I) has a hydrophobic amino acid in the first position, wherein the hydrophobic amino acid is selected from the group consisting of glycine, alanine, valine, leucine, isoleucine, proline, phenylalanine, methionine, and tryptophan; wherein the numbering of Zₙ starts at the N-terminal side.

31. The method according to claim 19, wherein Zₙ is KA_{q}K, with q being an integer of from 1 to 13, and wherein R¹ is a saturated, or unsaturated straight, branched or cyclic alkyl chain of 15 carbon atoms and R² is a saturated, or unsaturated straight, branched or cyclic alkyl chain of 16 carbon atoms.

32. The method according to claim 31, wherein q is an integer of from 1 to 7.

33. The method according to claim 19, wherein the compound according to Formula (I), or a salt or solvate thereof, is comprised in a pharmaceutical composition, wherein said pharmaceutical composition further comprises a pharmaceutically acceptable carrier, diluent, or excipient.

34. The method according to claim 19, wherein the disease is a viral infection.

35. The method according to claim 34, wherein the viral infection is caused by one or more viruses from the Flaviviridae family.

35. The method according to claim 19, wherein the disease is dengue.

36. The method according to claim 19, wherein the ADAM17-mediated disease is selected from the group consisting of ulcerative colitis, rheumatoid arthritis, cystic fibrosis, COPD, IPF, Crohn's disease, multiple sclerosis, and atherosclerosis.

37. The method according to claim 19, wherein the subject is a human.
